(19) United States
(12) Patent Application Publication
(10) Pub. No.: US 2011/0111978 A1
(43) Pub. Date: May 12, 2011

(54) DIVERSE CHEMICAL LIBRARIES BOUND TO SMALL PARTICLES WITH PARAMAGNETIC PROPERTIES

(75) Inventors: Egisto Boschetti, Croissy sur Seine (FR); Lee Lomas, Pleasanton, CA (US)

(73) Assignee: Bio-Rad Laboratories, Inc., Hercules, CA (US)

(21) Appl. No.: 13/004,281

(22) Filed: Jan. 11, 2011

Related U.S. Application Data
(62) Division of application No. 12/162,125, filed on Jul. 24, 2008.
(60) Provisional application No. 60/664,794, filed on Mar. 23, 2005.

(51) Int. Cl. C40B 30/04; C40B 50/14; B82Y 5/00

(52) U.S. Cl. 506/9; 506/30; 977/773

(57) ABSTRACT

The present invention provides diverse chemical libraries bound to small particle with paramagnetic properties. Typically, the chemical structures comprise a plurality of different chemical moieties, the particles are paramagnetic and have a diameter between about 100 nm and about 10 microns, the chemical structures bound to each particular particle have substantially the same structure and the combinatorial library comprises at least 100,000 different chemical structures.

This application is a divisional of co-pending application Ser. No. 12/162,125, filed Jul. 24, 2008, which is a U.S. National Phase of PCT/US2006/010647, filed Mar. 22, 2006, which claims the benefit of U.S. provisional patent application No. 60/664,794, filed Mar. 23, 2005, and international patent application number PCT/US2006/010647, filed Mar. 22, 2006 and U.S. application Ser. No. 11/388,181, filed on Mar. 22, 2006. The disclosures of all of the applications listed above are incorporated herein by reference in their entirety.

### FIELD OF THE INVENTION

The present invention relates to the fields of combinatorial chemistry, protein chemistry and biochemistry.

### BACKGROUND OF THE INVENTION

Large collections (e.g., libraries) of molecules have emerged as important tools for the successful identification of useful compounds. Such libraries are typically synthesized using combinatorial approaches as described further herein. A combinatorial library is a collection of multiple species of chemical compounds comprised of smaller subunits or monomers, such as a combinatorial peptide library comprised of amino acid residues or a combinatorial nucleic acid library comprised of nucleotides. Combinatorial libraries come in a variety of sizes, ranging from a few hundred to several million species of chemical compounds. A library of linear hexamer peptides made with 18 of the natural amino acids, for example, contains 34×10⁶ different chemical structures. When amino acid analogs and isomers are also included, the number of potential structures is practically limitless. The chemical approach also facilitates the synthesis of cyclic and branched peptides. There are also a variety of library types, including oligomeric and polymeric libraries comprised of compounds such as peptides, carbohydrates, nucleic acids, oligonucleotides, and small organic molecules, etc.

Libraries of thousands, even millions, of random oligopeptides have been prepared by chemical synthesis (Houghten et al., 1991, Nature 354:84-6), or gene expression (Marks et al., 1991, J Mol Biol 222:581-97), displayed on chromatographic supports (Lam et al., 1991, Nature 354:82-4), inside bacterial cells (Colas et al., 1996, Nature 380:548-550), on bacterial pili (Lu, 1990, Bio/Technology 13:366-372), or phage (Smith, 1985, Science 228:1315-7). Libraries of proteins (Ladner, U.S. Pat. No. 4,664,989), peptoids (Simon et al., 1992, Proc Natl Acad Sci USA 89:9367-71), nucleic acids (Ellington and Szostak, 1990, Nature 246:818-22), carbohydrates, and small organic molecules (Eichler et al., 1995, Med Res Rev 15:481-96) have also been prepared. In addition, cyclic peptides, peptide amides, peptide aldehydes, etc. were directly synthesized on solid supports (Barany et al., 1987, Int. J Peptide Protein Res 30:705-739; Fields et al., 1990, Int. J Peptide Protein Res 35:161-214; Lloyd-Williams et al, 1993, Tetrahedron 49:11065-11133; Wang, 1973, J Amer Chem Soc 95:1328; Barlos et al., 1989, Tetrahedron Letters 30:3947; Beebe et al., 1995, J Org Chem 60:4204; Rink, 1987, Tetrahedron Letters 28:3787; Rapp et al., in “Peptides 1988”, Proc. 20th European Peptide Symposium, Jung G. and Boyer E. (Eds.), Walker de Gruyter, Berlin, pp 199 1989].

To make a combinatorial library, a solid-phase support (resin) is reacted with one or more subunits of the compounds and with one or more numbers of reagents in a carefully controlled, predetermined sequence of chemical reactions. In other words, the library subunits are “grown” on the solid-phase support. Solid-phase supports are typically polymeric objects with surfaces that are functionalized to bind with subunits or monomers to form the compounds of the library. Synthesis of one library typically involves a large number of solid-phase supports. Solid-phase supports known in the art include, among others, polystyrene resin beads, cotton threads, and membrane sheets of polytetrafluoroethylene (“PTFE”).

Combinatorial libraries have a variety of uses, such as identifying and characterizing ligands capable of binding an acceptor molecule or mediating a biological activity of interest (Scott and Smith, 1990, Science 249:386-390; Salmon et al., 1993, Proc Natl Acad Sci USA 90:11708-11712;), binding to anti-peptide antibodies (Fodor et al., 1991, Science 251:767-773; Needles et al., 1993, Proc Natl Acad Sci USA 90:10700-10704; Valadon et al., 1996, J Mol Biol 261:11-22), screening for binding to a variety of targets including cellular proteins (Schmitz et al., 1996, J Mol Biol 260:664-677), viral proteins (Hong and Boulanger, 1995, EMBO J. 14:4714-4727), bacterial proteins (Jacobsson and Frykberg, 1995, Biotechniques 18:878-885), nucleic acids (Cheng et al., 1996, Gene 171:1-8), plastic (Siani et al., 1994, J Chem Inf Comput Sci 34:588-593), and molecules having biological function (Hammon et al., U.S. patent application No. 2004/0101830.

Another important use for large ligand libraries is in proteomics, more specifically, for reducing the range in concentration of analytes in a complex biological mixture, such as serum. This method, also referred to as “equalization,” involves exposing a solid phase-bound ligand library with proteins from a sample. When a large library is used, most or all of the proteins in the sample are bound by at least one unique ligand in the library. By limiting the size of the library used, that is, the actual number of total ligands, highly abundant proteins will saturate their ligands, while rare proteins will not. After washing away proteins for which there are insufficient ligands to binds, the retained proteins have a compressed range of concentrations—the relative amounts of the most abundant proteins is closer to that of the rare proteins. This method is described, for example, in EP 1 580 559 A1 (Boschetti). In performing this method, small volumes of a ligand library are useful when the sample to be “equalized” is only available in small quantities.

### SUMMARY OF THE INVENTION

It is an object of this invention to provide a solution to the problem of manipulating very small particles during split-couple-and-recombine combinatorial chemical synthesis useful for the analysis of complex protein mixtures and for purifying proteins. In one aspect of the present invention, a method involves providing small particles with paramagnetic properties on which the split-couple-and-recombine combinatorial chemical synthesis will be performed, and manipulating the particles through magnetism, e.g., using magnets.

In a preferred embodiment of the present invention, a method of making a combinatorial library of diverse chemical structures bound to particles is provided. This method comprises the step of performing a number of rounds of split-couple-and-recombine chemical synthesis with a collection of particles with paramagnetic properties having a diameter between about 100 nm and about 10 microns and a plurality of different chemical moieties, wherein each round of the split-couple-and-recombine chemical synthesis adds a chemical moiety to the chemical structure, and involves magnetically manipulating the particle with paramagnetic properties, and wherein the number of rounds suffices to assemble a library having a diversity of at least 100,000 unique chemical structures.

In certain embodiments, the particles with paramagnetic properties have a diameter between about 300 nm and about 5 microns or between about 1 micron and 3 microns.

Many chemical structures can be used to practice methods of the invention and produce compositions of the invention. Preferred chemical structures are peptides, oligonucleotides, oligosaccharides or synthetic organic molecules.

The library has a diversity of large number of unique chemical structures. Preferred libraries of the present invention have a diversity of at least 1 million unique chemical structures and even more preferred the library has a size of at least 100,000,000 chemical structures.

In embodiments where the chemical structures are peptides, the library has a diversity of at least 3 million unique peptides, preferably at least 64 million unique peptides.

Preferred are libraries that comprise substantially all of the members of a combinatorial library.

Using the particles with paramagnetic properties having a diameter between about 100 nm and about 10 microns, in a preferred embodiment, a library and in particular a peptide library, is less than about 100 microliters.

The particles with paramagnetic properties can be made in different ways. In one embodiment, the particles with paramagnetic properties comprise a polymeric material with a paramagnetic material embedded therein. The particles with paramagnetic properties can also comprise porous particles wherein a paramagnetic material is lodged in the pores of these particles.

In another aspect of the present invention, a library of diverse chemical structures bound to a collection of particles with paramagnetic properties having a diameter between about 100 nm and about 10 microns is provided. The chemical structures of such libraries comprise a plurality of different chemical moieties and the chemical structures bound to each individual particle with paramagnetic properties have substantially the same structure. Typically, such a library has a diversity of at least 100,000 unique chemical structures.

In a preferred embodiment, the particles are substantially monodisperse, the chemical structures are peptides and the library has a diversity of at least 300,000 unique peptides. Also preferred are libraries having a diversity of at least 3,000,000 unique peptides, preferable a diversity of at least 30,000,000 unique peptides, more preferable a diversity of at least 64,000,000 unique peptides, and even more preferable a diversity of at least 100,000,000 unique peptides. A preferred library is a library that comprises substantially all of the members of the combinatorial library.

The particles may comprise various crosslinked synthetic or natural polymers. Preferred are particles wherein the crosslinked synthetic or natural polymer is polyacrylate, polyvinyl, polystyrene, nylon, polyurethane or a polysaccharide.

In another aspect of the present invention, a library of diverse chemical structures bound to a collection of particles with paramagnetic properties having a diameter between about 100 nm and about 10 microns is provided, wherein the chemical structures comprise a plurality of different chemical moieties, the library has a diversity of at least 100,000 unique chemical structures and each particular particle has a majority of the diversity of the chemical structures bound thereto.

The present invention also provides kits. Preferred kits of the present invention comprise a library of the invention. Kits of the invention, for example, can be used to decrease the range of concentration of analytes in a mixture, to detect analytes in a mixture or for purifying a protein. Accordingly, the kits comprise one or more instructions for using the library to decrease the range of concentration of analytes in a mixture, for detecting analytes in a mixture or for purifying a protein. Optionally, a kit also comprises a container containing a buffer. Additional kit embodiments of the present invention include optional functional components that would allow one of ordinary skill in the art to perforin any of the method variations described herein.

The compositions of the present invention are useful to practice many different methods. A preferred use of a composition of the present invention is in a method for decreasing the range of concentration of different analyte species in a mixture. This method comprises the following steps: (a) providing a first sample comprising a plurality of different analyte species present in the first sample in a first range of concentrations; (b) contacting the first sample with an amount of a library of diverse chemical structures bound to a collection of particle with paramagnetic properties having a diameter between about 100 nm and about 10 microns, wherein the chemical structures comprise a plurality of different chemical moieties and the chemical structures bound to each individual particle with paramagnetic properties have substantially the same structure and the combinatorial library has a diversity of at least 100,000 unique chemical structures; (c) capturing amounts of the different analyte species from the first sample with the different chemical structures and removing unbound analyte species; and (d) isolating the captured analyte species from the chemical structures to produce a second sample comprising a plurality of different analyte species present in the second sample in a second range of concentrations; wherein the amount of the library is selected to capture amounts of the different analyte species so that the second range of concentrations is less than the first range of concentrations.

In one aspect of this method, isolation of the captured analyte species may comprise a step-wise elution to produce a plurality of aliquots.

Optionally, this method comprises the step of detecting the isolated analyte species. Detection can be by mass spectrometry or electrophoresis.

In a preferred embodiment, isolating the captured analyte comprises eluting the analytes from the particles onto a biochip with an adsorbant surface, wherein the adsorbant surface binds the analytes from the eluate.

In still another aspect of the present invention, a method for detecting analytes in a mixture is provided. In a preferred embodiment, this method comprises the steps of (a) providing a first sample comprising a plurality of different analyte species present in the first sample in a first range of concentrations; (b) contacting the first sample with an amount of a library of diverse chemical structures bound to a collection of particles with paramagnetic properties having a diameter between about 100 nm and about 10 microns, wherein the chemical structures comprise a plurality of different chemical moieties and the chemical structures bound to each individual particle with paramagnetic properties have substantially the same structure and the combinatorial library has a diversity of at least 100,000 unique chemical structures; (c) capturing amounts of the different analyte species from the first sample with the different chemical structures and removing unbound analyte species; (d) placing the particles with captured analytes into a mass spectrometer; and (e) detecting the captured analytes by laser desorption mass spectrometry.

Further, the present invention provides a method for purifying a target protein group. In a preferred embodiment, this method comprises the steps of: (a) contacting a sample comprising at least 95% of the target protein group and at most 5% of contaminating proteins with a library of diverse chemical structures bound to a collection of particle with paramagnetic properties having a diameter between about 100 nm and about 10 microns, wherein the chemical structures comprise a plurality of different chemical moieties and the chemical structures bound to each individual particle with paramagnetic properties have substantially the same structure and the combinatorial library has a diversity of at least 100,000 unique chemical structures in an amount sufficient to bind contaminating proteins and a minority of the target protein group; (b) binding the contaminating proteins and the minority of the target protein group to the library of chemical structures; (c) separating the unbound target protein group from the contaminating proteins and target protein group bound to the library of chemical structures; and (d) collecting the unbound target protein group from the sample; whereby the collected target protein group is more pure than the target protein group in the sample.

### BRIEF DESCRIPTION OF THE DRAWINGS

FIG. 1 depicts an SDS-PAGE analysis showing the result of a comparative analysis of an equalization method using regular beads (lane b) and magnetized beads (lane c). Lane a shows a molecular marker. Details are provided in Example 1.

FIG. 2 depicts an SDS-PAGE analysis of serum samples treated with magnetized solid phase hexapeptide ligand library (lane c) and regular beads (lane b; data from Example 1) and initial human serum proteins (lane a). Details are provided in Example 2.

FIG. 3 depicts a SELDI MS analysis of samples from 14 different serum treatment trials. The ProteinChip Array used was Q10. The molecular weight range shown is from about 5 kDa to about 20 kDa Details are provided in Example 3.

### DETAILED DESCRIPTION OF THE INVENTION

Biological samples, such as serum, cerebrospinal fluid and others, may be available to the researcher only in quantities of no more than a few milliliters. In screening experiments, it is preferred to use as little of this precious material as possible. One method of analyzing biological samples involves exposing the sample to a diverse chemical library bound to particles made, e.g., by a “split-couple-and-recombine” method. However, typically the particles used to make such libraries are in the 40 micron to 100 micron size range. A complete combinatorial library of hexapeptides of the 20 amino acids has a diversity of about 64 million unique peptide species. Attached to beads having a 40 micron to 100 micron size range, the library has a volume of about 16 milliliters. Generally the beads are loaded with a minimum of ten volumes of serum, corresponding to 160 mL or 9600 mg of proteins. To deal with serum volumes of 100 μL, 10 μL of ligand library would be required. Such a library would have a diversity of only about 30,000 unique hexapeptide species, which is not optimal for capturing the diversity of proteins in a complex biological sample such as serum. Additionally when sampling 10 μL of such a library from a large stock of material composed of several dozen of millions of combinations, each individual sample would be different from another. Consequently the final result could be of questionable reproducibility.

One approach to solving this problem is to use very small particles, for example in the range of 200 nanometers to 10 microns in diameter. In the first case 10 μl, of these beads would comprise 1.25×10¹² beads; in the second case the same volume would comprise about 10⁷ beads. However, beads of such size are extremely difficult to work with. In particular, split-couple-and-recombine methods of combinatorial chemistry typically involve performing chemical synthesis in flow-through columns followed by a filtration to separate solvents and excess reagents. Small particles would become stuck in filters in these columns, making it impractical to wash the particles and to pool them after chemical coupling. Centrifugation, as an alternative method of separation, is labor-intensive and time consuming.

This invention provides a solution to the problem of manipulating very small particles during split-couple-and-recombine combinatorial chemical synthesis. The method involves providing small particles with paramagnetic properties on which the chemical synthesis will be performed, and manipulating the particles through magnetism, e.g., using magnets.

This invention also provides libraries of particle-bound ligands in which a majority or substantially all of the unique members of the library are attached to a each individual particle.

### I. SMALL PARTICLES WITH PARAMAGNETIC PROPERTIES

A. Paramagnetic and Non-Paramagnetic Materials

The particles of this invention have paramagnetic properties. That is, the particles have atomic magnetic dipoles that align with an external magnetic field. Accordingly, the particles of this invention are attracted by magnets and can attract like normal magnets when subject to a magnetic field. The particles are generally monodisperse, their diameter can range between 100 and 1000 nm. During the manipulation these beads stay in suspension; they are then separated by a magnetic field. “Substantially monodisperse” means that the standard deviation in the range of diameters of the particles is no more than 2%.

The particles with paramagnetic properties of this invention generally comprise a paramagnetic material and a non-paramagetic material to which the chemical structures are chemically bound, generally covalently.

The paramagnetic material is constituted of very fine particles of mineral oxides with paramagnetic properties such as magnetite (a mixed iron oxide), hematite (an iron oxide), chromite (a salt of iron and chrome) and all other material attracted by a permanent magnet of electromagnet. Also ferrites such as iron tritetraoxide (Fe₃O₄), γ-sesquioxide (γ-Fe₂O₃), MnZn-ferrite, NiZn-ferrite, YFe-garnet, GaFe-garnet, Ba-ferrite, and Sr-ferrite; metals such as iron, manganese, cobalt, nickel, and chromium; alloys of iron, manganese, cobalt, nickel, and the like, but not limited thereto, can be used. The preferred material is magnetite because its availability and low cost. It is supplied as particles of different size, dry or as an aqueous stabilized suspension.

These particles are dispersed within the polymeric network and confer to the entire particle the property to be attracted by a permanent magnet or an electromagnet.

The non-paramagnetic material on which chemical structures are attached are made of polymeric materials. Among the most common polymeric materials are cross-linked acrylates, polystyrene, polyurethane, polyvinyl, nylon, and polysaccharides. More specifically, these polymeric materials include organic polymers produced by polymerization of a polymerizable monomer: the monomer including styrenic polymerizable monomers such as styrene, α-methylstyrene, β-methylstyrene, o-methylstyrene, m-methylstyrene, p-methylstyrene, 2,4-dimethylstyrene, p-n-butylstyrene, p-t-butylstyrene, p-n-hexylstyrene, p-n-octylstyrene, p-n-nonylstyrene, p-n-decylstyrene, p-n-dodecylstyrene, p-methoxystyrene, and p-phenylstyrene; acrylic polymerizable monomers such as methyl acrylate, ethyl acrylate, n-propyl acrylate, isopropyl acrylate, n-butyl acrylate, isobutyl acrylate, t-butyl acrylate, n-amyl acrylate, n-hexyl acrylate, 2-ethylhexyl acrylate, n-octyl acrylate, n-nonyl acrylate, cyclohexyl acrylate, benzyl acrylate, dimethylphosphatoethyl acrylate, diethylphosphatoethyl acrylate, dibutylphosphatoethyl acrylate, and 2-benzoyloxyethyl acrylate; methacrylic polymerizable monomer such as methyl methacrylate, ethyl methacrylate, n-propyl methacrylate, isopropyl, methacrylate, n-butyl methacrylate, isobutyl methacrylate, t-butyl methacrylate, n-amyl methacrylate, n-hexyl methacrylate, 2-ethylhexyl methacrylate, n-octyl methacrylate, n-nonyl methacrylate, diethylphosphatoethyl methacrylate, acrylamide, methacrylamide and derivatives; dibutylphosphatoethyl methacrylate; methylene-.aliphatic monocarboxylic acid esters; vinyl polymerizable monomer such as vinyl esters, vinyl acetate, vinyl propionate, vinyl benzoate, vinyl butyrate, vinyl benzoate, and vinyl formate; vinyl ethers such as vinyl methyl ether, vinyl ethyl ether, and vinyl isobutyl ether; and vinyl ketones such as vinyl methyl ketone, vinyl hexyl ketone, and vinyl isopropyl ketone. Other examples of the polymeric structures are those made of inorganic solids, including clay minerals such as kaolinite, bentonite, talc, and mica; metal oxides such as alumina, titanium dioxide, and zinc oxide; insoluble inorganic salts such as silica gel, hydroxyapatite, and calcium phosphate gel; metals such as gold, silver, platinum, and copper; and semiconductor compounds such as GaAs, GaP, and ZnS. The material is not limited thereto. The polymeric structure may be used in combination of two or more thereof.

These non-paramagnetic polymeric networks could be compact or porous. In the first case the external surface area is used for the interaction with analytes, in the second case all the porous structure would be used for molecular interaction if the pores are large enough for a free diffusion of analytes.

B. Size of Microparticulate Solid Support

A preferred embodiment of the present invention utilizes small, beaded, microparticulate solid supports that are less than 10 μm, preferably between 200 nanometers and 10 microns in diameter, between 300 nm and 5 microns or between 1 and 3 microns in diameter. (Diameter of a non-spherical particle refers to the length in the longest dimension.) Microparticulate solid supports are desirable because they possess increased surface area to volume ratio compared to the larger bead. Microparticulate solid supports also decrease the volume of support necessary to contain a full combinatorial library, thereby allowing more complex and efficient libraries to be used.

C. Making Small Beaded Material with Paramagnetic Properties

Particles with paramagnetic properties useful for this invention are available from several commercial suppliers. These include, for example, Dynal (Invitrogen) (Carlsbad, Calif.), Ademtech (Pessac France—superparamagnetic nanoparticles) and Spherotech (Libertyville, Ill.).

Small beaded materials with paramagnetic properties of the present invention can be made using several methods.

In one embodiment of the present invention a particle or an aggregate of particles of magnetite can be encapsulated within a polymeric external layer on which combinatorial ligands can then be attached.

In another embodiment of the present invention, a paramagnetic material can be obtained by loading a pre-existing non-paramagnetic porous polymeric bead with an aqueous colloidal suspension of a paramagnetic particle, such as magnetite. These later paramagnetic particles progressively diffuse into the porous polymeric bead and are trapped as they form internal aggregates within the pore structure. The excess paramagnetic material that is not trapped within the polymer bead is then washed away using appropriate solvents. This ‘loading’ of paramagnetic material can be completed either before or after the ligands of a combinatorial library are attached to the polymeric bead.

In another embodiment, the particle with paramagnetic properties can be made by mixing a paramagnetic material with a polymer or monomers, and polymerizing or cross-linking the polymers or monomers. In the first case a solution of acrylic or vinyl monomers is added with small paramagnetic materials and kept in suspension by appropriate stirring. The solution is then poured to a non miscible solvent so as to obtain a suspension of droplets. The size of the droplets and their distribution depends on the methods of stirring. Once the droplet suspension has reached the expected size, monomers are polymerized and droplets turn into small beads. This method is referred to “emulsion polymerization.” The particles of the paramagnetic material are consequently trapped within the polymeric network. In the second case a solution of polysaccharide (e.g. agarose, dextran) is added with small paramagnetic materials (e.g., particles) and kept in suspension by appropriate stirring while adding appropriate crosslinking agents (e.g. bisepoxyranes, divinylsulfone) and the pH adjusted so that to get conditions of crosslinking. The solution of polysaccharide with particles in suspension is then poured to a non miscible solvent so that to obtain a suspension of droplets. The size of the droplets and their distribution depends on the methods of stirring. Once the droplet suspension has reached the expected size, the suspension is left at a pre-determined temperature until the crosslinking reaction is achieved. Small aqueous droplets turn progressively into small beads. The particles of paramagnetic material are consequently trapped within the polymeric network conferring paramagnetic properties to the obtained material.

D. Solid Supports

The suitability of solid support materials for use in the present invention in particular for synthesizing peptide libraries may be evaluated against the following criteria: (a) the ability to synthesize peptides on the solid support (the solid support should be stable for all the solvents used in the synthesis of the combinatorial peptide library); (b) the solid support should contain a free amino group, or a suitable stable but cleavable linker (however, it should be noted that a cleavable linker is not required); (c) the solid support should be mechanically stable during synthesis, screening and handling; (d) the size of the solid support should be large enough to allow manual handling, or whatever alternative handling means is contemplated; (e) the peptide capacity of the bead should be at least about 10 pmole of peptide per bead, or whatever lower limit is rendered feasible by advances in sequencing and detection technology (a capacity of about 100 pmole is preferable); and (f) the solid support should display a low degree of non-specific adsorption of ligands of choice and of proteins in general. It will be recognized by a person of ordinary skill in the art that these criteria should not be considered absolute requirements.

Acceptable solid supports for use in the present invention can vary widely. A solid support can be porous or nonporous, but is preferably porous. It can be continuous or non-continuous, flexible or nonflexible. A solid support can be made of a variety of materials including ceramic, glassy, metallic, organic polymeric materials, or combinations thereof.

The shape of the microparticulate support may be in a shape of a film of a plastic material such as—polyethylene terephthalate (PET), diacetate, triacetate, cellophane, celluloid, polycarbonate, polyimide, polyvinyl chloride, polyvinylidene chloride, polyacrylates, polyethylene, polypropylene, and polyesters; a porous film of a polymer such as polyvinyl chloride, polyvinyl alcohol, acetylcellulose, polycarbonate, nylon, polypropylene, polyethylene, and Teflon; a wood plate; a glass plate; a silicon substrate; a cloth formed from a material such as cotton, rayon, acrylic fiber, silk, and polyester-fiber; and a paper sheet such as wood free paper, medium-quality paper, art paper, bond paper, regenerated paper, baryta paper, cast-coated paper, corrugated board paper, and resin-coated paper. Naturally the shape of the carrier is not limited thereto. The material in a shape of a film or sheet may have a smooth surface or a rough surface insofar as the magnetic substance can be held thereon.

Preferred solid supports include organic polymeric supports, such as particulate or beaded supports, polyacrylamide and mineral supports such as silicates and metal oxides can also be used. Particularly preferred embodiments include solid supports in the form of spherical or irregularly-shaped beads or particles.

Porous materials are useful because they provide large surface areas. The porous support can be synthetic or natural, organic or inorganic. Suitable solid supports are very similar to chromatographic sorbents for protein separation with a porous structure have pores of a diameter of at least about 1.0 nanometer (nm) and a pore volume of at least about 0.1 cubic centimeter/gram (cm³/g). Preferably, the pore diameter is at least about 30 nm because larger pores will be less restrictive to diffusion. Preferably, the pore volume is at least about 0.5 cm³/g for greater potential capacity due to greater surface area surrounding the pores. Preferred porous supports include particulate or beaded supports such as agarose, hydrophilic polyacrylates, polystyrene, mineral oxides, including spherical and irregular-shaped beads and particles.

For significant advantage, the solid supports for chemical structures are preferably hydrophilic. Preferably, the hydrophilic polymers are water swellable to allow for greater infiltration of analytes. Examples of such supports include natural polysaccharides such as cellulose, modified celluloses, agarose, cross-linked dextrans, amino-modified cross-linked dextrans, guar gums, modified guar gums, xanthan gums, locust bean gums and hydrogels. Other examples include cross-linked synthetic hydrophilic polymers such as polyacrylamide, polyacrylates, polyvinyl alcohol (PVA) and modified polyethylene glycols. Preferred polymeric material is the one compatible with solvents used to construct the combinatorial libraries according to their composition.

Generally, the particle with paramagnetic properties comprises reactive groups, such as amines or carboxyls, or reactive groups generally well known for the preparation of affinity chromatography supports onto which chemical moieties can be coupled.

Non-reacted cross-linking groups on the surface may be reacted with a small chemical such a mercaptoethanol to prevent further reactivity. In addition, surfaces may be further treated to prevent non-specific adhesion of protein.

The microparticulate solid support includes paramagnetic beads allowing for an easy one-step separation of unbound target protein group and proteins bound to the chemical structures coupled to the paramagnetic beads.

### II. LIBRARY OF CHEMICAL STRUCTURES

A library of chemical structures used in this invention comprises a collection of at least 100,000 different chemical structures. In certain embodiments the library of chemical structures comprises at least, 300,000, 1,000,000, 3,000,000, 10,000,000, 50,000,000, or at least 100,000,000 unique chemical structures. Preferably, at least one chemical structure in the library recognizes each analyte in the mixture to be analyzed. Preferably, the library of chemical structures includes at least as many different chemical structures as there are analytes in the sample.

Typically, and as described in detail below, library of chemical structures are coupled to an insoluble solid support or particulate material. Each solid support or insoluble particle preferably carries several copies of the same chemical structure, with each particle type coupling a different chemical structure.

Library of chemical structures of the present invention may be produced using any technique known to those of skill in the art. For example, library of chemical structures may be chemically synthesized, harvested from a natural source or, in the case of library of chemical structures that are bio-organic polymers, produced using recombinant techniques. However, in a preferred embodiment, the chemical structures are produced through combinatorial synthesis using the well-known “split-couple-and-recombine” method.

Chemical structures may be purchased pre-coupled to the solid supports, or may be indirectly attached or directly immobilized on the solid support using standard methods (see, for example, Harlow and Lane, Antibodies, Cold Spring Harbor Laboratory, Cold Spring Harbor, N.Y. (1988); Biancala et al., Letters in Peptide Science 2000, 7(291):297; MacBeath et al., Science 2000, 289:1760-1763; Cass et al., ed., Proceedings of the Thirteenth American Peptide Symposium; Leiden, Escom, 975-979 (1994); U.S. Pat. No. 5,576,220; Cook et al., Tetrahedron Letters 1994, 35:6777-6780; and Fodor et al., Science 1991, 251(4995):767-773).

A. Combinatorial Libraries

In one embodiment of this invention the library of chemical structures is a combinatorial library or portion thereof. A combinatorial chemical library is a collection of compounds generated by either chemical synthesis or biological synthesis, by combining a number of chemical “building blocks” in all possible combinations. For example, a complete linear combinatorial chemical library, such as a polypeptide library, is formed by combining a set of chemical building blocks (amino acids) in every possible way for a given compound length (i.e., the number of amino acids in a polypeptide compound). As an example, if the number of building blocks is 5 and the construct is composed of five members, the number of possible linear combinations is of 5⁵ or 3,125 members. In this case the building blocks (A, B, C, D and E) are assembled linearly such as: A-A-A-A-A; A-A-A-A-B; A-A-A-A-C; A-A-A-B-A; A-A-A-B-B; A-A-A-B-C; A-A-B-A-A; A-A-B-A-B; A-A-B-A-C; . . . ; E-E-E-E-C; E-E-E-E-D; E-E-E-E-E. “Substantially all” of the members of a combinatorial library is at least 95% of the unique members of the library.

Another form of combinatorial library is scaffold-based. These constructs are based of a single central molecule or core, comprising positions that can be selectively and/or sequentially substituted by building blocks. An example is given by trichloro-triazine (three selectively temperature-dependent substitutable positions) on which several substituents can be attached. If the number of substituents is three, the number of possible combinations is 10. It is also possible to consider the relative positioning of each substituent; in this case the number of combinations is larger.

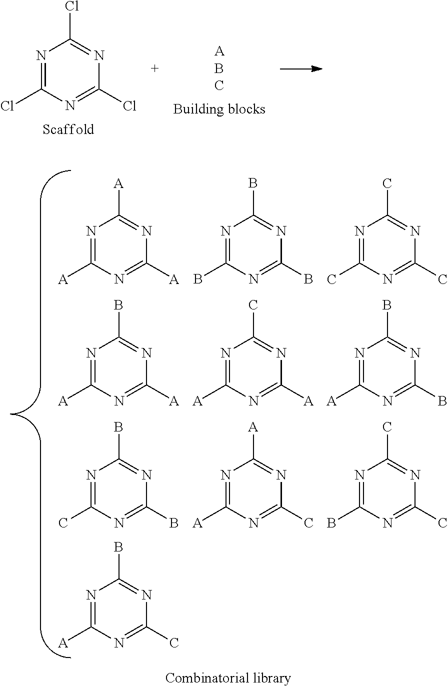

Another example of scaffold is given by lysine where the three substitutable positions (carboxyl, alpha-amine and epsilon-amine) can be selectively protected thus selectively substitutable by binding blocks.

As a third level it is possible to combine linear combinatorial libraries with scaffold-based libraries where substituents of this latter are combinatorial linear sequences.

Millions of chemical compounds can be synthesized through such combinatorial mixing of chemical building blocks. For peptide chemical structures, the length is preferably limited to 15, 10, 8, 6 or 4 amino acids. Polynucleotide chemical structures of the invention have preferred lengths of at least 4, more preferably 6, 8, 10, 15, or at least 20 nucleotides. Oligosaccharides are preferably at least 5 monosaccharide units in length, more preferably 8, 10, 15, 20, 25 or more monosaccharide units.

Combinatorial libraries may be complete or incomplete. Complete combinatorial libraries of biopolymers are those libraries containing a representative of every possible permutation of monomers for a given polymer length and composition. Incomplete libraries are those libraries lacking one or more possible permutation of monomers for a given polymer length.

Combinatorial and synthetic chemistry techniques well-known in the art can generate libraries containing millions of members (Lam et al., Nature 354: 82-84 (1991) and International (PCT) Patent Application WO 92/00091), each having a unique structure. A library of linear hexamer ligands made with 18 of the natural amino acids, for example, contains 34×10⁶ different structures, a library made with 20 amino acids, for example, contains 64×10⁶ different structures. When amino acid analogs and isomers are also included, the number of potential structures is practically limitless. Members of a combinatorial library can be synthesized on or coupled to a solid support, such as a bead, with each bead essentially having millions of copies of a library member on its surface. As different beads may be coupled to different library members and the total number of beads used to couple the library members is large, the potential number of different molecules capable of binding to the bead-coupled library members is enormous.

Hammond et al., US 2003/0212253 (Nov. 13, 2003) describes combinatorial libraries along the following lines. Peptide chemical structure libraries may be synthesized from amino acids that provide increased stability relative to the natural amino acids. For example, cysteine, methionine and tryptophan may be omitted from the library and unnatural amino acids such as 2-naphylalanine and norleucine included. The N-terminal amino acid may be a D-isomer or may be acetylated to provide greater biochemical stability in the presence of amino-peptidases. The chemical structure density must be sufficient to provide sufficient binding for the target molecule, but not so high that the chemical structures interact with themselves rather than the target molecule. A chemical structure density of 0.1 μmole-500 μmole per gram of dry weight of support is desired and more preferably a chemical structure density of 10 μmole-100 μmmole per gram of support is desired. A 6-mer peptide library was synthesized onto Toyopearl-AF Amino 650M resin (Tosoh USA, Grove City, Ohio). The size of the resin beads ranged from 60-130 mm per bead. Initial substitution of the starting resin was achieved by coupling of a mixture of Fmoc-Ala-OH and Boc-Ala-OH (1:3.8 molar ratio). After coupling, the Boc protecting group was removed with neat TFA in full. The resulting deprotected amino groups were then acetylated. Peptide chains were assembled via the remaining Fmoc-Ala-OH sites on the resin bead. Standard Fmoc synthetic strategies were employed. In one embodiment a typical experiment, six grams of Fmoc-Ala-(Ac-Ala-)Toyopearl Resin was deprotected with 20% piperidine/DMF (2×20 min), then washed with DMF (8 times) and equally divided into 18 separate reaction vessels. In each separate vessel, a single Fmoc-amino acid was coupled to the resin (BOP/NMM, 5-10 told excess) for 4-7 hours. The individual resins were washed and combined using the “split/mix” library technique (Furka et al., Int. J. Peptide Protein Res., 37, 487-493 (1991); Lam et al., Nature, 354, 82-84 (1991); International Patent Application WO 92/00091 (1992); U.S. Pat. No. 5,010,175; U.S. Pat. No. 5,133,866; and U.S. Pat. No. 5,498,538). The cycle of deprotection and coupling was repeated until the amino acid sequence was completed (six cycles for a hexamer library). The final Fmoc was removed from peptide resins using 20% piperidine/DMF in separate reaction vessels during the last coupling cycle. Side-chain protecting groups were removed with TFA treatment for 2 hours. Resins were washed extensively and dried under a vacuum. Peptide densities achieved were typically in the range of 0.06-0.12 mmol/g of resin.

Sequencing and peptide composition of peptide ligand-resin bead complexes were confirmed, and the degree of substitution of the resin was calculated by quantitative amino acid analysis at Commonwealth Biotechnologies, Inc., Richmond, Va. Sequencing was performed at Protein Technologies Laboratories, Texas A&M University, by Edman degradation using a Hewlett PackardG1005A.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Combinatorial libraries and especially peptide libraries can be chemically modified by the introduction of various substituents. For instance a peptide library with a terminal primary amine group can be chemically substituted with a number of molecules conferring peculiar additional properties. Exposed amino groups (terminal and side lysine chains) can be reacted with a large number of molecules having a reactive moiety such as epoxy, aldehyde, carboxyl, anhydride, acylchloride, isocyanate, vinylsulfone, tosylates, lactones and others. When the reactive moiety reacts with the primary amino group of the library it add to the library and additional structure. The library is thus endcapped with chemical of biochemical functions that may be complementary to the initial library.

For instance a primary amino terminal peptide is reacted with succinyl anhydride, the introduction of a terminal carboxyl group is obtained at the bottom of a spacer of two methylene groups. The overall property of the resulting library changes from its initial dominant cationic character to a net anionic character This change unambiguously induce a different behavior for the reduction of the concentration range of components of a complex mixture. Primary amine terminal libraries can also be advantageously mixed with carboxyl terminal libraries with potentially a larger field of applicability.

Another way to modify the available primary amines of a peptide library is to introduce a terminal sugar; in this case better hydrophilicity is obtained along with the possibility to capture species that have an affinity for sugars that is enhanced by the presence of a structure from the combinatorial peptide chains.

In another example to the terminal primary amino groups chelating agents can be attached. When these chemical functions are added with transition metal ions, the behavior of the entire library is modified and addresses more specifically proteins that can have metal ion interactions. In this case the library would possess an additional feature that can be exploited after protein adsorption by a selective desorption using specific displacing agents such as chelating agents and more specifically EDTA.

Chemical reaction to make derivatives are not only limited to combinatorial peptides, but also to all other libraries such as combinatorial oligonucleotides and oligosaccharides.

1. Small Organic Molecules

In a preferred embodiment of the present invention, the method comprises the step of contacting a sample with a library of chemical structures, wherein the library is a combinatorial library of small organic molecules.

Accordingly, small molecules are also contemplated as library of chemical structures for use in the methods and kits of the present invention. Typically, small organic molecules have properties that allow for ionic, hydrophobic or affinity interaction with an analyte. Libraries of small organic molecules include chemical groups traditionally used in chromatographic processes such as mono-, di- and tri-methyl amino ethyl groups, mono-, di- and tri-ethyl amino ethyl groups, sulphonyl, phosphoryl, phenyl, carboxymethyl groups and the like. For example libraries may use benzodiazepines, (see, e.g. Bunin et al., Proc Natl Acad Sci USA 1994, 91:4708-4712) and peptoids (e.g. Simon et al., Proc Natl Acad Sci USA 1992, 89:9367-9371; Gilon et al., Biopolymers 1991, 31:745-750)). Peptoids are peptide analogs in which the peptide bond (—NHCO—) is replaced by an analogous structure, e.g., —NRCO—. In another embodiment, the chemical structure is a dye or a triazine derivative. This list is by no means exhaustive, as one of skill in the art will readily recognize thousands of chemical functional groups with ionic, hydrophobic or affinity properties compatible with use as library of chemical structures in the methods of the present invention.

In a preferred embodiment of the present invention, the combinatorial library of small organic molecules is covalently attached to a solid support, preferably a plurality of beads. As described further herein, attachment of the combinatorial library of small organic molecules to the solid support can be direct or via a linker.

2. Biopolymers

In a preferred embodiment of the present invention, the method comprises the step of contacting a sample with a library of chemical structures, wherein the library is a combinatorial library of biopolymers.

In one embodiment of the present invention, biopolymers are selected from the group consisting of polypeptides, polynucleotides, lipids and oligosaccharides.

For biopolymer library of chemical structures of the present invention, linear length is preferably between 4 and 50 monomeric units, in particular no more than 15, no more than 10, desirably 8, 7, 6, 5, 4 or 3 monomeric units. For peptide libraries, the length is preferably limited to no more than 15, 10, 8, 6 or 4 amino acids. Nucleic acid libraries have preferred lengths of at least 4, more preferably at least 6, 8, 10, 15, or at least 20 nucleotides. Oligosaccharides are preferably at least 5 monosaccharide units in length, more preferably at least 8, 10, 15, 20, 25 or more monosaccharide units.

In one embodiment of the present invention, the biopolymers are covalently attached to a solid support, preferably a plurality of beads. As described further herein, attachment of the combinatorial library of biopolymers to the solid support can be direct or via a linker.

a) Peptides

In a preferred embodiment of the present invention, a biopolymer is a peptide. Particularly preferred library of chemical structures comprise peptides having no more than 50, 40, 30, 25, 20, 15, 10, 8, 6 or 4 amino acids, as they are easily produced using recombinant or solid phase chemistry techniques. Moreover, peptide library of chemical structures may be produced in a manner that eases their use for the methods of the present invention. For example, peptides may be recombinantly produced as a phage display library where the peptide is presented as part of the phage coat (see, e.g., Tang et al., J Biochem 1997, 122(4):686-690). In this context, the peptides would be attached to a solid support, the phage. Other methods for generating libraries of peptide chemical structures suitable for use in the claimed invention are also well known to those of skill in the art, e.g., the “split, couple, and recombine” method (see, e.g., Furka et al., Int J Peptide Protein Res 1991, 37:487-493; Fodor et al., Science 1991, 251:767-773; Houghton et al., Nature 1991, 354:84-88; Lam et al., Nature 1991, 354:82-84; International Patent Application WO 92/00091; and U.S. Pat. Nos. 5,010,175, 5,133,866, and 5,498,538, all of which herewith are incorporated in their entirety by reference) or other approaches known in the art. The expression of peptide libraries also is described in Devlin et al., Science 1990, 249:404-406.

Combinatorial peptide libraries, such as combinatorial hexapeptide libraries may be synthesized using one or more of the twenty amino acids that are genetically encoded: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Of these, all save glycine are optically isomeric, however, only the L-form is found in humans. Nevertheless, the D-forms of these amino acids do have biological significance; D-Phe, for example, is a known analgesic. Thus, both D- and L-forms of these amino acids can be used as building blocks for a combinatorial peptide library.

Many other amino acids are also known and find use as building blocks for peptide libraries, including: 2-aminoadipic acid; 3-aminoadipic acid; beta-aminopropionic acid; 2-aminobutyric acid; 4-aminobutyric acid (piperidinic acid); 6-aminocaproic acid; 2-aminoheptanoic acid; 2-aminoisobutyric acid, 3-aminoisobutyric acid; 2-aminopimelic acid; 2,4-diaminobutyric acid; desmosine; 2,2′-diaminopimelic acid; 2,3-diaminopropionic acid; N-ethylglycine; N-ethylasparagine; hydroxylysine; allo-hydroxylysine; 3-hydroxyproline; 4-hydroxyproline; isodesmosine; allo-isoleucine; N-methylglycine (sarcosine); N-methylisoleucine; N-methylvaline; norvaline; norleucine; and ornithine.

Libraries of peptide chemical structures may be synthesized from amino acids that provide increased stability relative to the natural amino acids. For example, cysteine, methionine and tryptophan may be omitted from the library and unnatural amino acids such as 2-naphylalanine and norleucine included. The N-terminal amino acid may be a D-isomer or may be acetylated to provide greater biochemical stability in the presence of amino-peptidases. The library density must be sufficient to provide sufficient binding for an analyte, but not so high that the library of chemical structures interact with themselves rather than the analyte. A library density in the range of 0.1 μmole to 500 μmole per gram of dry weight of solid support is desired and more preferably a library density in the range of 10 μmole to 100 μmole per gram of solid support is desired. Other preferred ranges are 10 μmole to 100 μmole per ml of solid support.

In a standard “Merrifield” synthesis, a side chain-protected amino acid is coupled by its carboxy terminal to a support material, such as a microparticulate resin. A side chain and amino terminal protected amino acid reagent is added, and its carboxy terminal reacts with the exposed amino terminal of the insolubilized amino acid to form a peptide bond. The amino terminal of the resulting peptide is then deprotected, and a new amino acid reagent is added. The cycle is repeated until the desired peptide has been synthesized. For an overview of techniques, see Geisaw, 1991, Trends Biotechnol 9:294-95).

In the conventional application of this procedure, the amino acid reagent is made as pure as possible. However, if a mixture of peptides is desired, the amino acid reagent employed in one or more of the cycles may be a mixture of amino acids, and this mixture may be the same or different, from cycle to cycle. Thus, if Ala were coupled to the solid support, and a mixture of Glu, Cys, His and Phe were added, the dipeptides Ala-Glu, Ala-Cys, Ala-His and Ala-Phe will be formed.

A peptide library may consist essentially only of peptides of the same length, or it may include peptides of different length. The peptides of the library may include, at any variable residue position, any desired amino acid. Possible sets include, but are not limited to: (a) all of the genetically encoded amino acids, (b) all of the genetically encoded amino acids except cysteine (because of its ability to form disulfide crosslinks), (c) all of the genetically encoded amino acids, as well as their D-forms; (d) all naturally occurring amino acids (including, e.g., hydroxyproline); (e) all hydrophilic amino acids; (f) all hydrophobic amino acids; (g) all charged amino acids; (h) all uncharged amino acids; etc. The peptide library may include branched and/or cyclic peptides.

In some combinatorial peptide library embodiments, the peptides are expressed on the surface of a recombinant bacteriophage to produce large libraries. Using the “phage method” (Scott and Smith, Science 249:386-390, 1990; Cwirla, et al., Proc. Natl. Acad. Sci., 87:6378-6382, 1990; Devlin et al., Science, 49:404-406, 1990), very large libraries can be constructed (10⁶-10⁸ chemical entities). A second approach uses primarily chemical methods, of which the Geysen method (Geysen et al., Molecular Immunology 23:709-715, 1986; Geysen et al., J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

In a preferred embodiment of the present invention, the method comprises the step of contacting a sample with a library of chemical structures, wherein the library of chemical structures comprises an antibody library antibody libraries (see, e.g., Vaughn et al., Nature Biotechnology 1996, 14(3):309-314; PCT/US96/10287). In a preferred embodiment of the present invention, the method comprises the step of contacting a sample with an antibody library displayed on phage particles

b) Polynucleotides

Nucleic acids are another preferred biopolymer library of chemical structures. As with peptides, nucleic acids may be produced using synthetic or recombinant techniques well-known to those of skill in the art. The terms “polynucleotide,” “nucleic acid,” and “nucleic acid molecule” are used interchangeably herein and refer to the polymeric form of deoxyribonucleotides, ribonucleotides, and/or their analogs in either single stranded form, or a double-stranded helix. A nucleic acid molecule may also comprise modified nucleic acid molecules, such as methylated nucleic acid molecules and nucleic acid molecule analogs. Analogs of purines and pyrimidines are known in the art. Nucleic acids may be naturally occurring, e.g., DNA or RNA, or may be synthetic analogs, as known in the art. Such analogs may be preferred for use as chemical structures because of superior stability. Modifications in the native structure, including alterations in the backbone, sugars or heterocyclic bases, have been shown to increase intracellular stability and binding affinity. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 31-CH₂-5′-O-phosphonate and 3′-NH-5′-O-phosphoroamidate. Peptide nucleic acids replace the entire ribose phosphodiester backbone with a peptide linkage.

When the biopolymer is a nucleic acid, conventional DNA or RNA synthesis and sequencing methods may be employed. The usual bases are the purines adenine and guanine, and the pyrimidines thymidine (uracil for RNA) and cytosine. However, unusual bases, such as those following, may be incorporated into the synthesis or produced by post-synthesis treatment with mutagenic agents: 4-acetylcytidine, 5-(carboxyhydroxylmethyl)uridine, 2′-O-methylcytidine, 5-carboxymethylaminomethyl-2-thioridine, 5-carboxymethylaminomethyluridine, dihydrouridine, 2′-O-methylpseudouridine, beta,D-galactosylqueosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, beta,D-mannosylqueosine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-yl)N-methylcarbamoyl)threonine, uridine-5-oxyacetic acid methylester, uridine-5-oxyacetic acid, wybutoxosine, pseudouridine, queosine, 2-thiocytidine, 5-methyl-2-thiouridine.-2-thiouridine, 4-thiouridine, 5-methyluridine, N-((9-beta-D-ribofuranosylpurine-6-yl)carbamoyl)threonine, 2′-O-methyl-5-methyluridine, 2′-O-methyluridine, wybutosine, 3-(3-amino-3-carboxypropyl)uridine.

Preferable nucleic acid chemical structures are at least 4, more preferably at least 6, 8, 10, 15, or 20 nucleotides in length. Nucleic acid chemical structures include double stranded DNA or single stranded RNA molecules (e.g., aptamers) that bind to specific molecular targets, such as a protein or metabolite.

c) Oligosaccharides

A biopolymer can be an oligosaccharide. Thus, oligosaccharide chemical structures are also contemplated for use in the methods and kits of the invention. Oligosaccharide chemical structures are preferably at least 5 monosaccharide units in length, more preferably at least 8, 10, 15, 20, 25 or more monosaccharide units in length.

Monosaccharides in a polymeric carbohydrate library may be aldoses, ketoses, or derivatives. They may be tetroses, pentoses, hexoses or more complex sugars. They may be in the D- or the L-form. Suitable D-sugars include D-glyceraldehyde, D-erythrose, D-threose, D-arabinose, D-ribose, D-lyxose, D-xylose, D-glucose, D-mannose, D-altrose, D-allose, D-talose, D-galactose, D-idose, D-gulose, D-rhamnose, and D-fucose. Suitable L-sugars include the L-forms of the aforementioned D-sugars.

d) Lipids

A biopolymer can be a lipid. As used herein, the term “lipid” refers to a hydrophobic or amphipathic moiety. Thus, lipid chemical structures are also contemplated for use in the methods and kits of the invention. Suitable lipids include a C14 to C50 aliphatic, aryl, arylalkyl, arylalkenyl, or arylalkynyl moiety, which may include at least one heteroatom selected from the group consisting of nitrogen, sulfur, oxygen, and phosphorus. Other suitable lipids include a phosphoglyceride, a glycosylglyceride, a sphingolipid, a sterol, a phosphatidyl ethanolamine or a phosphatidyl propanolamine. Lipid chemical structures are preferably at least 5 units in length, more preferably at least 8, 10, 15, 20, 25, 50 or more units in length.

### III. ATTACHMENTS OF CHEMICAL STRUCTURES TO SOLID SUPPORT

A. Assembling Chemical Structures on Particle with Paramagnetic Properties Using “Split-Couple-and-Recombine” Methods

“Split-Couple-and-Recombine” is a well known method of combinatorial synthesis that involves a number of rounds of spitting solid supports into a plurality of aliquots; coupling a moiety, such as monomer, to the supports or to the chemical structures attached to the solid supports in previous rounds; and pooling the solid supports to allow mixing. Following is a description of the method in more detail.

A certain amount of magnetic beads of a diameter of less than 10 microns with appropriate linker is split into a number of groups containing equal amounts. The number of groups is the same as the number of building blocks that are to be used for the preparation of the library. For instance if an oligonucleotide library were to be made using the standard adenosine, thymidine, cytosine and guanidine nucleotides, the groups of beads would be four as the number of mononucleotides. The building blocks would be named “a”, “b”, “c” and “d. On the first group of beads the building bloc “a” will be attached. Building blocks “b”, “c” and “d” will be respectively attached on the second, third and fourth bead groups. Once the four distinct operations are achieved in suspension under gentle agitation, by-products and used solvents for the synthesis will be washed out.

This operation cannot be done by filtration because particles having a diameter of less than 10 microns are too small and will clog the filters. The present invention solves this problem by providing particles having paramagnetic properties and then manipulating these particles during the split-couple-and-recombine process using magnetic force. One mode of separating them is to maintain particles with paramagnetic properties within the synthesis vessel by means of an externally positioned permanent magnet and remove the solvent by simple rotation of the vessel to evacuate the liquid. Alternatively particles with paramagnetic properties can also be removed from the liquid solvents by introducing inside the suspension an activated electromagnet on which all paramagnetic materials will stick. Once washed extensively and removed from the final washing solution, the beads are mixed together. This operation is done by releasing captured paramagnetic particles by the electromagnet inside a common vessel. Beads will be released by a simple deactivation of the electromagnet. Once all together beads are mixed thoroughly with a classical stirrer and then split again into four equal groups. On the first group the building block “a” will be attached while building blocks “b”, “c”, and “d are respectively reacted with the second, third and fourth group of particles with paramagnetic properties. Similar operations as described above will follow: washing, recovery and mixing before re-splitting again. The number of iterations depends on the desired length of the ligand library. Typically with amino acids the most common number of building blocks used is 6 (hexapeptide) while with oligonucleotides it may vary from 15 to 30.

The solid support can be derivatized with a fully prepared library of chemical structures by attaching a previously prepared library of chemical structures to the solid support. Alternatively, the library of chemical structures may be formed on the solid support by attaching a precursor molecule to the solid support and subsequently adding additional precursor molecules to the growing chain bound to the solid support by the first precursor molecule. This mechanism of building the adsorbent on the solid support is particularly useful when the chemical structure is a polymer, particularly a biopolymer such as a polypeptide, polynucleotide or polysaccharide molecule. A biopolymer chemical structures can be provided by successively adding monomeric components (e.g., amino acids, nucleotides or simple sugars) to a first monomeric component attached to the solid support using methods known in the art. See, e.g., U.S. Pat. No. 5,445,934 (Fodor et al.), incorporated herewith in its entirety by reference.

The “diversity” of the library is the expected number of unique chemical structure formulae in the library.

The “size” of the library is the estimated number of chemical structure molecules in it. The size depends on the initial number of building blocks and the length of the final combinatorial ligand. In all cases employing split-couple-and-recombine synthesis, the number of beads necessary to prepare a library must exceed the final number of diversomers. If for example the library is made using 15 building blocks and the final ligands is a 9mer, the final library will be composed of 15⁹ structures (this corresponds to about 4×10¹⁹ structures or diversomers). In this case if particles with paramagnetic properties have a diameter of 6 μm (each μL of packed particles with paramagnetic properties corresponds to 4.6×10⁶ beads) the minimum volume of beads to be used must be higher than 10 mL of particles with paramagnetic properties. In the case of hexapeptides made using 20 different amino acids attached on particles with paramagnetic properties of 2.8 μm diameter, the volume of particles with paramagnetic properties must exceed 1.5 μL. In certain embodiments, the number of beads in the library will suffice so that at least 2 different beads, at least 4 different beads or at least 8 different beads each comprise the same unique chemical structure. For example, a bead library of about 250 million beads can include four beads each comprising the same chemical structure of a 64 million-member library.

As few as one and as many as 10, 100, 1,000, 10,000, 1,000,000, 3,000,000, 10,000,000, 1,000,000,000 or more chemical structures may be coupled to a single solid support. In preferred embodiments the solid support is in the form of beads, with a single, different, chemical structure type bound to each bead. For example in a peptide chemical structure library, peptides representing one possible permutation of amino acids would be bound to one bead, peptides representing another possible permutation to another bead, and so on.

Chemical structures may be coupled to a solid support using reversible or non-reversible reactions. For example, non-reversible reactions may be made using a support that includes at least one reactive functional group, such as a hydroxyl, carboxyl, sulfhydryl, or amino group that chemically binds to the chemical structures, optionally through a spacer group. Suitable functional groups include N-hydroxysuccinimide esters, sulfonyl esters, iodoacetyl groups, aldehydes, epoxy, imidazolyl carbamates, and cyanogen bromide and other halogen-activated supports. Such functional groups can be provided to a support by a variety of known techniques. For example, a glass surface can be derivatized with aminopropyl triethoxysilane in a known manner. In some embodiments, chemical structures are coupled to a solid support during synthesis, as is known to those of skill in the art (e.g., solid phase peptide and nucleic acid synthesis).

Alternatively, reversible interactions between a solid support and a chemical structure may be made using linker moieties associated with the solid support and/or the chemical structures. A variety of linker moieties suitable for use with the present invention are known, some of which are discussed herein. Use of linker moieties for coupling diverse agents is well known to one of ordinary skill in the art, who can apply this common knowledge to form solid support/chemical structure couplings suitable for use in the present invention with no more that routine experimentation.

In another embodiment, each different chemical structure can be coupled to a different solid support. This is the case, for example, when a combinatorial library is built on beads using the split-couple-and-recombine method. Alternatively, a collection of chemical structures can be coupled to a pool of beads, so that each bead has a number of different chemical structures attached. This can be done, for example, by creating a combinatorial library on a first set of supports, cleaving the chemical structures from the supports and re-coupling them to a second collection of supports.

In a preferred aspect the present invention provides a method for making a combinatorial library of diverse chemical structures bound to a collection of particles with paramagnetic properties and having a diameter between about 100 nm and about 10 microns, comprising the steps of: (a) providing a plurality of different chemical moieties; (b) performing a first round of split-pool-and-recombine chemical synthesis with the collection of particles having an activated group, wherein the first round of the split-pool-and-recombine chemical synthesis adds a first chemical moiety of the plurality of different chemical moieties to the activated group on the collection of particles; (c) magnetically manipulating the collection of particles with paramagnetic properties; and (d) performing a second round of split-pool-and-recombine chemical synthesis wherein the second round of the split-pool-and-recombine chemical synthesis adds a second chemical moiety of the plurality of different chemical moieties to the first chemical moiety attached to the activated group on the collection of particles; wherein the number of rounds of split-pool-and-recombine chemical syntheses suffices to assemble a library having a diversity of at least 100,000 unique chemical structures.

B. Particles with Paramagnetic Properties in which a Majority of the Diversity of the Chemical Structures is Bound to Each Individual Particle with Paramagnetic Properties

In another embodiment of the invention, the chemical structures of the library are attached to the particles after they are synthesized. In this way each particular particle will have a plurality of different chemical structures attached, and a single particle can have a majority or substantially all of the members of a combinatorial library attached. In one method of making, 2 microliters of particles with paramagnetic properties having reactive groups on a polymeric moiety are washed repeatedly with a carbonate buffer at pH 9.5. The liquid phase is separated from the particles by means of a magnetic field produced by a permanent magnet. Once the washing step is done, the particles are contacted with 1500 micrograms of soluble hexpeptide library. The suspension is shaken overnight at room temperature to promote the chemical coupling of peptides on beds via their primary available amino groups. The excess of reactive groups on the particles are destroyed adding lysine or ethanol amine.

### IV. REDUCING RELATIVE ANALYTE CONCENTRATIONS IN A SAMPLE

A. Interacting Forces

While not wishing to be limited by theory, it is believed that a variety of interactions influence how analytes are captured on solid-phase bound libraries of chemical structures. Proteins are captured by magnetic bead ligand library as a function of the structure of the ligand attached on each bead. By definition each ligand is composed of structures that carry complex conformations and collection of different ligands is very diverse. For example, if the library is composed of hexapeptides, the building block (amino acids) comprise aromatic rings, heterocycles, positive and negative charges, hydrophobic moieties.

The types of interactions that are established between a protein and its ligand partner are similar to forces that stabilize the conformation of macromolecules. They are generally one order of magnitude less than that of covalent bonds. These weak interactions involve atoms or groups of atoms attracted or repelled to minimize the energy of conformation. They can be grouped into: ion-ion, hydrogen bonding, dipole-dipole, dispersion and hydrophobic interactions. The permanent dipole-permanent dipole; permanent dipole-induced dipole and induced dipole-induced dipole interactions are collective listed under the name of van-der-Waals interactions. Weak existing induced dipole-induced dipole interactions are those called attractive London dispersion forces.

These attraction forces are dependent on distance between partners with the energies being inversely proportional to the distance or to some power of the distance separating the atomic arrangement of protein epitope from the atomic conformation of the combinatorial ligand. As the power of the inverse distance dependency increases, the interaction approaches zero very rapidly. Directly opposing this kind of attraction, is steric repulsion, which does not allow two atoms to occupy the same space at the same time. Together, the attractive dispersion and repulsive exclusion interactions define an optimum distance separating two atoms at which the energy of interactions is at minimum.

The energies associated with long-range interactions (e.g., charge-charge, charge-dipole) are dependent on the environmental medium. The interaction between two charged atoms, for example, becomes shielded in a polar medium and is therefore weakened. The expression for the energy of long-range interactions are all inversely related to the dielectric constant of the medium and are thus weakened in a highly polarizable medium such as water. The composition of the medium additionally affects other important weak interactions, such as hydrogen bonds and hydrophobic interactions. This is why, when capturing proteins with the hexameric ligand library, the process is conducted under native physiological conditions of pH and of ionic. Among strong interaction forces generated by the positioning of atoms on both protein and ligands (e.g. peptides) are hydrogen bonding and hydrophobic associations.

There are a large variety of hydrogen bondings that can favour the interaction of the hexameric ligands with native proteins: interaction between ═NH and the oxygen of a carbonyl along the peptide bonds of the α-helix; between ═NH and a —OH group; between ═NH and the imidazole ring; between ═NH and the oxygen of a carboxyl and, finally, between two —OH groups (such as those of Ser, Thr and Tyr).

Hydrophobic associations are generated by the concomitant presence of water repellent structures close each other. A number of amino acids comprise such structures: isoleucine, valine and leucine are major examples. Also classified by hydropathy index among relatively hydrophobic aminoacids are tryptophane, tyrosine and phenylalanine probably due to their aromatic ring.

B. Suitable Test Samples

Test samples of the present invention may be in any form that allows analytes present in the test sample to be contacted with binding moieties of the present invention, as described herein. Suitable test samples include gases, powders, liquids, suspensions, emulsions, permeable or pulverized solids, and the like. Preferably test solutions are liquids. Test samples may be taken directly from a source and used in the methods of the present invention without any preliminary manipulation. For example, a water sample may be taken directly from an aquifer and treated directly using the methods described herein.

Alternatively, the original sample may be prepared in a variety of ways to enhance its suitability for testing. Such sample preparations include depletion of certain analytes, concentrating, grinding, extracting, percolating and the like. For example, solid samples may be pulverized to a powder, and then extracted using an aqueous or organic solvent. The extract from the powder may then be subjected to the methods of the present invention. Gaseous samples may be bubbled or percolated through a solution to dissolve and/or concentrate components of the gas in a liquid prior to subjecting the liquid to methods of the present invention.

Test samples preferably contain at least 1000, 100,000, 1,000,000, 10,000,000 or more analytes of interest. In some circumstances, test samples suitable for manipulation using the methods of the present invention may include hundreds or thousands of analytes of interest. Preferably, the concentrations of analytes present in the test sample spans at least an order of magnitude, more preferably at least two, three, four or more orders of magnitude. Once subjected to the methods of the present invention, this concentration range for analytes detectable by at least one detection method will be decreased by at least a factor of two, more preferably a factor of 10, 20, 50, 100, 1000 or more.

For example, serum is known to contain analytes present in a concentration range of mg/ml for the most abundant down to pg/ml for the most rare. This is a concentration range of at least 10⁹ orders of magnitude. However, after reduction in concentration range using the methods of this invention, the range in concentrations can be reduced by at least one to four or more orders of magnitude.

Test samples may be collected using any suitable method. For example, environmental samples may be collected by dipping, picking, scooping, sucking, or trapping. Biological samples may be collected by swabbing, scraping, withdrawing surgically or with a hypodermic needle, and the like. The collection method in each instance is highly dependent upon the sample source and the situation, with many alternative suitable techniques of collection well-known to those of skill in the art.

Test samples may be taken from any source that potentially includes analytes of interest including environmental samples such as air, water, dirt, extracts and the like. A preferred test sample of the present is a biological sample, preferably a biological fluid. Biological samples that can be manipulated with the present invention include amniotic fluid, blood, cerebrospinal fluid, intraarticular fluid, intraocular fluid, lymphatic fluid, milk, perspiration plasma, saliva semen, seminal plasma, serum, sputum, synovial fluid, tears, umbilical cord fluid, urine, biopsy homogenate, cell culture fluid, cell extracts, cell homogenate, conditioned medium, fermentation broth, tissue homogenate and derivatives of these. Analytes of interest in biological samples include proteins, lipids, nucleic acids and polysaccharides. More particularly, analytes of interest are cellular metabolites that are normally present in the animal, or are associated with a disease or infectious state such as a cancer, a viral infection, a parasitic infection, a bacterial infection and the like. Particularly interesting analytes are those that are markers for cellular stress. Analytes indicating that the animal is under stress are an early indicator of a number of disease states, including certain mental illnesses, myocardial infarction and infection.

Analytes of interest also include those that are foreign to the animal, but found in tissue(s) of the animal. Particularly interesting analytes in this regard include therapeutic drugs including antibiotics, many of which exist as different enantiomers and toxins that may be produced by infecting organisms, or sequestered in an animal from the environment. Samples can be, for example, egg white or E. coli extracts.

C. Capturing Analytes from a Test Sample Using Libraries of Chemical Structures

Analytes present in a test sample are captured by contacting the test sample with the binding moieties under conditions that allow each binding moiety to couple with its corresponding analyte. As inferred above, binding moieties may be contacted with the test sample directly, or the binding moieties may be first attached to a solid support, such as a dipstick, SELDI probe, or insoluble polymeric bead, membrane or powder.

These procedures also can be carried out using the paramagnetic properties of the particles to manipulate them. That is, after mixing the particles with a sample and incubating, the particles with analytes attached can be separated from the liquid by applying a magnetic force to attract the particles and separate them from liquid. The liquid can be removed by, e.g., pipette. Then, new liquid can be added for washing, mixed with the particles, and the particles can be separated from the wash, again by applying magnetic force.

In the case in which the binding moieties are, part of a bead library, the ratio of paramagnetic bead volume to sample volume for a complex sample such as serum can be between, for example, 1:150 and 1:1. The smaller the ratio of beads to sample, the greater the ability to increase the relative concentration of low abundance or rare analyte species. A preferred constant ratio of bead:sample volume is about 1:10.

Contacting the binding moiety with the test sample may be accomplished by mixing the two, swabbing the test sample onto the binding moiety, flowing the test sample over the solid support having binding moieties attached thereto, and other methods that would be obvious to those of ordinary skill in the art. The binding moieties and the analytes are kept in contact for a time sufficient to allow the binding moieties to reach binding equilibrium with the sample. Under typical laboratory conditions this is at least 10 minutes.

D. Removing Unbound Analytes

A feature of the present invention is that treatment of analytes according to the methods described herein preferably concentrates and partially purifies bound analyte in addition to reducing the variance between analyte concentrations. Implementation of this feature to the fullest includes optionally washing any unbound analytes from the analyte bound to the binding moieties on the solid support.

Washing away unbound analyte is preferably performed by contacting the analyte bound to the binding moiety with a mild wash solution. The mild wash solution is designed to remove contaminants and unbound analytes frequently found in the test sample originally containing the analyte. Typically a wash solution will be at a physiologic pH and ionic strength and the wash will be conducted under ambient conditions of temperature and pressure.

Formulation of wash solutions suitable for use in the present invention can be performed by one of skill in the art without undue experimentation. Methods for removing contaminants, including low stringency washing methods, are published, for example in V. Thulasiraman et al., Electrophoresis, 26, (2005), 3561-3571; Scopes, Protein Purification: Principles and Practice (1982); Ausubel, et al. (1987 and periodic supplements); Current Protocols in Molecular Biology; Deutscher (1990) “Guide to Protein Purification” in Methods in Enzymology vol. 182, and other volumes in this series.

E. Isolating Captured Analytes from Binding Moieties

The existence of well defined protein-ligand interactions especially when they are associated within a single structure, play an important role in the magnetic bead capturing process. It is by the analysis and knowledge of these forces that it is possible to distinguish eluting agents that can be used for the recovery for captured proteins out of a very complex mixture such as serum.

Having considered the importance of interacting forces, it is possible to devise eluting agents. By that way it is possible to either desorb proteins all together or to desorb then sequentially according to their dominant type of interaction. For ion-ion dominating interactions (this is the case when the peptide ligand is mostly or totally composed of acidic amino acids such as aspartic acid or glutamic acid, proteins can be eluted by a salt solution such as 1 M sodium chloride, as customarily done in ion-exchange chromatography. This process, in general, should allow recovery of proteins in a native form, thus permitting further monitoring. A similar effect as the presence of salt can also be obtained by disrupting ionic bonds by an appropriate electric field, a process that also maintain protein integrity.

To disrupt mildly hydrophobic interactions between proteins and ligand of particles with paramagnetic properties, 50% ethylene glycol could be used (likewise in affinity chromatography). However, for strong hydrophobic associations (hexapeptides mostly composed of leucine, isoleucine or valine) hydro-organic mixtures comprising isopropanol, acetonitrile and similar solvents in water are preferred. Another type of protein elution is 200 mM glycine-HCl, at pH 2.5: this eluent is typically adopted to disrupt tenacious interactions possibly related to conformational structures, such as those occurring between antigens and antibodies in an immuno-affinity column. These interactions are the result of many synergistic forces present at the same time. In this case very low pHs contribute to significantly deform protein epitopes reducing thus the interaction then weakened by a relatively high ionic strength.

Mixtures of 2 M thiourea, 7 M urea, 4% CHAPS in water appear to be an excellent eluant for proteins adsorbed onto peptide libraries. This is a mixed-mode eluant, able to disrupt simultaneously hydrogen bondings as well as hydrophobic associations releasing thus a vast population of proteins. Concentrated aqueous solutions of urea at acidic or alkaline pHs are also used with an almost quantitative protein desorption efficacy. Finally, for eluting protein en masse, one could use 6 M guanidine HCl (GuHCl), pH 6. Due to its strong chaotropic effect and its high ionic strength this solution is considered as a general eluant, able to disrupt all bonds and reduce all protein to random polymer coils. GuHCl can be used as the sole elution step, if all proteins have to be desorbed at once, or as the final step, at the end of the cascade of sequential elutions. (See, e.g., Scopes, Protein Purification: Principles and Practice (1982); and Deutscher (1990) “Guide to Protein Purification” in Methods in Enzymology vol. 182, and other volumes in this series)

A typical sequence to desorb proteins by groups from particles with paramagnetic properties is the use first of an increase of ionic strength by the addition of sodium chloride. As a second eluent an acidic solution of 100-300 mM glycine-HCl, pH 2.2-2.6 followed by a hydro-organic mixture of isopropanol-acetonitrile-water. Finally in the case of some more proteins are still adsorbed on beads the use of 9M urea at pH 3.3 is recommended.

Examples of suitable elution buffers include those that modify surface charge of an analyte and/or binding moiety, such as pH buffer solutions. pH buffer solutions used to disrupt surface charge through modification of acidity preferably are strong buffers, sufficient to maintain the pH of a solution in the acidic range, i.e., at a pH less than 7, preferably less than 6.8, 6.5, 6.0, 5.5, 5.0, 4.0 or 3.0; or in the basic range at a pH greater than 7, preferably greater than 7.5, 8.0, 8.3, 8.5, 9.0, 9.3, 10.0 or 11.0. In certain embodiments, the elution buffer can comprise 9 M urea at pH 3, 9 M urea at pH 11 or a mixture of 6.66% MeCN/13.33% IPA/79.2% H20/0.8% TFA. The selection of one method versus another depends on the analytical method used for the equalized sample.

Alternatively, solutions of high salt concentration having sufficient ionic strength to mask charge characteristics of the analyte and/or binding moiety may be used. Salts having multi-valent ions are particularly preferred in this regard, e.g., sulphates and phosphates with alkali earth or transition metal counterions, although salts dissociating to one or more mono-valent are also suitable for use in the present invention, provided that the ionic strength of the resulting solution is at least 0.1, preferably 0.25, 0.3, 0.35, 0.4, 0.5, 0.75, 1.0 mol l-1 or higher. By way of example, many protein analyte/binding moiety interactions are sensitive to alterations of the ionic strength of their environment. Therefore, analyte may be isolated from the binding moiety by contacting the bound analyte with a salt solution, preferably an inorganic salt solution such as sodium chloride. This may be accomplished using a variety of methods including bathing, soaking, or dipping a solid support to which the analyte is bound into the elution buffer, or by rinsing, spraying, or washing the elution buffer over the solid support. Such treatments will release the analyte from the binding moiety coupled to the solid support. The analyte may then be recovered from the elution buffer.

Chaotropic agents, such as guanidine and urea, disrupt the structure of the water envelope surrounding the binding moiety and the bound analyte, causing dissociation of complex between the analyte and binding moiety. Chaotropic salt solutions suitable for use as elution buffers of the present invention are application specific and can be formulated by one of skill in the art through routine experimentation. For example, a suitable chaotropic elution buffer may contain urea or guanidine ranging in concentration from 0.1 to 9 M.

Detergent-based elution buffers modify the selectivity of the affinity molecule with respect to surface tension and molecular complex structure. Suitable detergents for use as elution buffers include both ionic and nonionic detergents. Non-ionic detergents disrupt hydrophobic interactions between molecules by modifying the dielectric constant of a solution, whereas ionic detergents generally coat receptive molecules in a manner that imparts a uniform charge, causing the coated molecule to repel like-coated molecules. For example, the ionic detergent sodium dodecyl sulphate (SDS) coats proteins in a manner that imparts a uniform negative charge. Examples of non-ionic detergents include Triton X-100, TWEEN, NP-40 and Octyl-glycoside. Examples of zwitterionic detergents include CHAPS.

Another class of detergent-like compounds that disrupt hydrophobic interactions through modification of a solution's dielectric constant includes ethylene glycol, propylene glycol and organic solvents such as ethanol, propanol, acetonitrile, and glycerol.

One buffer of the present invention includes a matrix material suitable for use in a mass spectrometer. A matrix material may be included in the elution buffer. Some embodiments of the invention may optionally include eluting analyte(s) from binding moieties directly to mass spectrometer probes, such as protein or biochips. In other embodiments of the invention the matrix may be mixed with analyte(s) after elution from binding moieties. Still other embodiments include eluting analytes directly to SEND or SEAC/SEND protein chips that include an energy absorbing matrix predisposed on the protein chip. In these latter embodiments, there is no need for additional matrix material to be present in the elution buffer.

Other elution buffers suitable for the present invention include combinations of buffer components mentioned above. Elution buffers formulated from two or more of the foregoing elution buffer components are capable of modifying the selectivity of molecular interaction between subunits of a complex based on multiple elution characteristics.

In one embodiment, the captured analytes are eluted with a elution buffer in continuous gradient or a step gradient. For example, a first elution buffer can be used that elutes only lightly adsorbed analytes. A next buffer can be used that elutes more strongly bound analytes, and so on. In this way, subsets of the analytes can be eluted into different aliquots.

Analytes isolated using the present invention will have a range of concentrations of analytes or concentration variance between analytes that is less than the range of concentrations of analytes or concentration variance originally present in the test sample. For example, after manipulation using the methods of the present invention, isolated analytes with have a range of concentrations of analytes or concentration variance from other isolated analytes that is decreased by at least a factor of two, more preferably a factor of 10, 20, 25, 50, 100, 1000 or more, from the concentration variance between the same analytes present in the test sample prior to subjecting the test sample to any of the methods described herein. Preferably, the method of the invention is performed with a minimal amount of elution buffer, to ensure that the concentration of isolated analyte in the elution buffer is maximized. More preferably, the concentration of at least one isolated analyte will be higher in the elution buffer than previously in the test sample.

After isolating the captured analytes, the analytes may be further processed by concentration or fractionation based on some chemical or physical property such as molecular weight, isoelectric point or affinity to a chemical or biochemical ligand. Fractionation methods for nucleic acids, proteins, lipids and polysaccharides are well-known in the art and are discussed in, for example, Scopes, Protein Purification: Principles and Practice (1982); Sambrook et al., Molecular Cloning—A Laboratory Manual (2nd ed.) Vol. 1-3, Cold Spring Harbor Laboratory, Cold Spring Harbor Press, N.Y., (Sambrook) (1989); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel).

F. Detecting Isolated Analytes

After analytes have been eluted and isolated free of binding moieties, the analyte may be detected, quantified or otherwise characterized using any technique available to those of ordinary skill in the art. A feature of applying the analysis techniques of the present invention to complex test samples, is the dynamic reduction of variance in analyte concentrations for isolated analytes relative to the large range in analyte concentration found in the original test sample. This reduction in analyte concentration range allows a much larger percentage of analytes found in the original test sample to be detected and characterized without recalibrating the detection device than would be available for analyte detection using the original test sample itself. The actual reduction in analyte concentration range achieved is dependent on a variety of factors including the nature of the original test sample, and the nature and diversity of the binding moieties used. Generally, the reduction in analyte concentration variance using the techniques described herein is sufficient to allow at least 25% more preferably at least 30%, 40%, 50%, 60%, 70%, 75% or 80% of the analytes isolated to be detected without instrument re-calibration. Ideally, the present invention allows at least 90%, 95%, 98% or more of the analytes isolated to be detected without instrument re-calibration.

Detecting analytes isolated using the techniques described herein may be accomplished using any suitable method known to one of ordinary skill in the art. For example, colorimetric assays using dyes are widely available. Alternatively, detection may be accomplished spectroscopically. Spectroscopic detectors rely on a change in refractive index; ultraviolet and/or visible light absorption, or fluorescence after excitation with a suitable wavelength to detect reaction components. Exemplary detection methods include fluorimetry, absorbance, reflectance, and transmittance spectroscopy. Other examples of detection are based on the use of antibodies (e.g., ELISA and Western blotting). Changes in birefringence, refractive index, or diffraction may also be used to monitor complex formation or reaction progression. Particularly useful techniques for detecting molecular interactions include surface plasmon resonance, ellipsometry, resonant mirror techniques, grating-coupled waveguide techniques, and multi-polar resonance spectroscopy. These techniques and others are well known and can readily be applied to the present invention by one skilled in the art, without undue experimentation. Many of these methods and others may be found for example, in “Spectrochemical Analysis” Ingle, J. D. and Crouch, S. R., Prentice Hall Publ. (1988) and “Analytical Chemistry” Vol. 72, No. 17.

A preferred method of detection is by mass spectroscopy. Mass spectroscopy techniques include, but are not limited to ionization (I) techniques such as matrix assisted laser desorption (MALDI), continuous or pulsed electrospray (ESI) and related methods (e.g., IONSPRAY or THERMOSPRAY), or massive cluster impact (MCI); these ion sources can be matched with detection formats including linear or non-linear reflection time-of-flight (TOF), single or multiple quadropole, single or multiple magnetic sector, Fourier Transform ion cyclotron resonance (FTICR), ion trap, and combinations thereof (e.g., ion-trap/time-of-flight). For ionization, numerous matrix/wavelength combinations (MALDI) or solvent combinations (ESI) can be employed. Subattomole levels of analyte have been detected, for example, using ESI (Valaskovic, G. A. et al., (1996) Science 273:1199-1202) or MALDI (Li, L. et al., (1996) J. Am. Chem. Soc. 118:1662-1663) mass spectrometry. ES mass spectrometry has been introduced by Fenn et al. (J. Phys. Chem. 88, 4451-59 (1984); PCT Application No. WO 90/14148) and current applications are summarized in recent review articles (R. D. Smith et al., Anal. Chem. 62, 882-89 (1990) and B. Ardrey, Electrospray Mass Spectrometry, Spectroscopy Europe, 4, 10-18 (1992)). MALDI-TOF mass spectrometry has been introduced by Hillenkamp et al. (“Matrix Assisted UV-Laser Desorption/Ionization: A New Approach to Mass Spectrometry of Large Biomolecules,” Biological Mass Spectrometry (Burlingame and McCloskey, editors), Elsevier Science Publishers, Amsterdam, pp. 49-60, 1990). With ESI, the determination of molecular weights in femtomole amounts of sample is very accurate due to the presence of multiple ion peaks that may be used for the mass calculation. A preferred analysis method of the present invention utilizes Surfaces Enhanced for Laser Desorption/Ionization (SELDI), as discussed for example in U.S. Pat. No. 6,020,208. Mass spectroscopy is a particularly preferred method of detection in those embodiments of the invention where elution of analytes directly onto a mass spectrometer probe or biochip occurs, or where the elution buffer contains a matrix material or is combined with a matrix material after elution of analytes from the binding moieties.

Another different mode of eluting captured proteins by combinatorial beads with paramagnetic properties can be associated with the analysis of the proteins. For instance when the size of the beads is small enough to have all ligand diversity within a volume of few μL, a sample of particles with paramagnetic properties associated with proteins can be directly loaded on a MALDI probe or on a ProteinChip array spot. The addition of the matrix (in the presence of solvents and acids) weakens the interaction of proteins with ligands and a laser fired on this mixture will ionized proteins which can consequently be detected by mass spectrometry.

Another method of detection widely used is electrophoresis separation based on one or more physical properties of the analyte(s) of interest. A particularly preferred embodiment for analysis of polypeptide and protein analytes is two-dimensional electrophoresis. A preferred application separates the analyte by isoelectric point in the first dimension, and by size in the second dimension. Methods for electrophoretic analysis of analytes vary widely with the analyte being studied, but techniques for identifying a particular electrophoretic method suitable for a given analyte are well known to those of skill in the art.

### V. PROTEIN PURIFICATION USING PARAMAGNETIC BEAD LIBRARIES

Very often contaminating proteins whose properties are not known are co-purified to a certain extent with a target protein and are very difficult to remove from the target protein. In the case of therapeutical protein solutions, for example, even trace amounts of contaminating proteins may have a disastrous effect on a patient to whom such therapeutical protein is administered. Such effects include severe allergic or immunological reactions. Often these effects are caused by contaminating proteins that are derived from eukaryotic or prokaryotic cells that are used to recombinantly express the therapeutical protein. These contaminating proteins are known as HCPs (Host Cell Proteins). HCPs, by definition, are very diverse and using methods of the prior art cannot be removed in a single process. Therefore their elimination is contingent upon a series of steps that also contribute to the reduction of the overall yield of the therapeutical protein of interest. Thus, it is a further object of the present invention to provide methods for the purification a protein of interest using the compositions described herein.

A. Contacting a Sample with and Binding a Sample to a Library of Chemical Structures

The present invention provides methods for purifying a target protein group. These methods comprise the steps of (a) contacting a sample comprising at least 95% of the target protein group and at most 5% of contaminating proteins with a library of chemical structures having at least 100 different chemical structures in an amount sufficient to bind contaminating proteins and a minority of the target protein group and (b) binding the contaminating proteins and the minority of the target protein group to the library of chemical structures.

Once again, particles with paramagnetic properties can be manipulated during the procedure with magnetic force to enable washing the particles and removing liquid, without losing the particles in the process.

When introduced to a sample containing a diversity of analytes, the chemical structures will bind various contaminants in the sample, such as contaminating proteins. Abundant analytes, such as the target protein group of interest, will be present in amounts far in excess of the amount necessary to saturate the capacity of their respective chemical structures. Therefore, a high percentage of the total amount of these abundant analytes will remain unbound and only a minority will bind to the chemical structures. Conversely, the lesser amounts of trace analytes, such as the contaminating proteins, means that these proteins will not saturate all of their available chemical structures. Therefore, the majority of the starting amount of the contaminating proteins will bind to their respective chemical structures.

Analytes, target protein groups and contaminating proteins, present in a sample are contacted with a library of chemical structures having at least 100,000 different chemical structures under conditions that allow each chemical structure to bind to its corresponding analyte if present in the sample. Generally, a sample is contacted with a library of chemical structures under conditions that allow binding of contaminating proteins and the minority of the target protein group to the chemical structures. The conditions under which a target protein group is purified will vary according to various parameters, including the inherent properties of the target protein group, the properties of the contaminating proteins, etc.

Contacting a sample with a library of chemical structures can be accomplished in a variety of ways. In a preferred method, the sample is mixed with the paramagnetic material and incubated for sufficient time to allow the contaminants to bind to the chemical structures. Then, the particle with paramagnetic properties, with the contaminants bound, are isolated from the solution using magnetic force. The solution is separated from the particles, and comprises purified protein.

Typically, the sample and the chemical structures are present in a binding buffer. Non-limiting examples of suitable binding buffers include a solution containing 50 mM sodium phosphate and 0.15 M NaCl, pH 7; a solution containing 50 mM sodium phosphate and 0.15 M NaCl, pH 8; and the like. Suitable binding buffers include, e.g., Tris-based buffers, borate-based buffers, phosphate-based buffers, imidazole, HEPES, PIPES, MOPS, MOPSO, MES, TES, acetate, citrate, succinate and the like.

Examples of suitable binding buffers include those that modify surface charge of an analyte and/or chemical structures, such as pH buffer solutions. pH buffer solutions preferably are strong buffers, sufficient to maintain the pH of a solution in the acidic range, i.e., at a pH less than 7, preferably less than 6.8, 6.5, 6.0, 5.5, 5.0, 4.0 or 3.0; or in the basic range at a pH greater than 7, preferably greater than 7.5, 8.0, 8.3, 8.5, 9.0, 9.3, 10.0 or 11.0. The pH conditions suitable for purifying a target protein group from a sample comprising the target protein group and contaminating proteins range from about 3.5 to about 11, from about 4.0 to about 10.0, from about 4.5 to about 9.5, from about 5.0 to about 9.0, from about 5.5 to about 8.5, from about 6.0 to about 8.0, or from about 6.5 to about 7.5. Typically, binding buffers have a pH range of about 6.5 to about 7.5. In an alternative embodiment of the present invention, binding buffers have a pH range of about 6.5 to about 8.5.

Alternatively, binding buffers of various salt concentrations may be used. Exemplary NaCl salt concentrations suitable for purifying a target protein group from a sample comprising the target protein group and contaminating proteins range from about 0.01 M NaCl to about 3 M NaCl, from about 0.05 M NaCl to about 1.5 M NaCl, from about 0.1 M NaCl to about 1.0 M NaCl, or from about 0.2 M NaCl to about 0.5 M NaCl. Preferred binding buffers have a salt concentration in the range of about 0 M to about 0.25 M. Other suitable salts in binding buffers are KCl or NaHOAc.

Other binding buffers suitable for the present invention include combinations of buffer components mentioned above. Binding buffers formulated from two or more of the foregoing binding buffer components are capable of modifying the selectivity of molecular interaction between contaminating proteins and chemical structures.

As will be appreciated by the ordinary skilled in the art, temperature conditions for protein purification may vary depending on the properties of the target protein group of interest to be purified. Typically, temperature conditions suitable for purifying a target protein group from a sample comprising the target protein group and contaminating proteins range from about 4° C. to about 40° C., from about 15° C. to about 40° C., from about 20° C. to about 37° C., or from about 22° C. to about 25° C. Typical temperature conditions are in the range from about 4° C. to about 25° C. One preferred temperature is about 4° C.

Contacting a sample with a library of chemical structures and binding of analytes to the chemical structures is done for a period of time sufficient for binding contaminating proteins and the minority of the target protein to the library of chemical structures. Typically, the library of chemical structures and the sample comprising the target protein group and the contaminating proteins are incubated together for at least about 10 min., usually at least about 20 min., more usually for at least about 30 min., more usually for at least about 60 min. Incubation time may also be for several hours, for example up to 12 hrs, but typically does not exceed about 1 hr. When the methods of the present invention are performed, for example, using a column, the time for contacting a sample with a library of chemical structures is referred to as residence time. A typical residence time range is from about 1 minute to about 20 minutes.

Once analytes have bound to the chemical structures, it may be desirable to elute the analytes for additional analyses. Among efficient elution buffers are those described in Table 1. They can be used singularly or according to a predetermined sequence (e.g., eluents that act on ion exchange effect first, followed by eluents capable to disassemble hydrophobic associations, etc.).

TABLE 1
Scheme of different elution protocols for proteins
adsorbed onto solid phase peptide library

| Eluting agent | Composition | Dissociated bonds |
|---|---|---|
| Salt | 1M Sodium chloride | Ionic interactions |
| Glycols | 50% ethylene glycol | Mildly hydrophobic associations |
| | in water | |
| Acidic pH | 200 mM Glycine-HCl pH 2.5 | Hydrogen bonding, conformation |
| | | changes |
| Dissociating-detergent | 2M thiourea-7M urea-4% | Mixed mode, hydrophobic |
| agents | CHAPS | associations, hydrogen bonding |
| Denaturant | 6M Guanidine-HCl pH 6 | All types of interactions |
| Hydro-organic | Acetonitrile (6.6)-isopropanol | Strong hydrophobic associations |
| | (33.3)-trifluoroacetic acid (0.5)- | |
| | water (49.5) | |
| Acidic dissociating | 9M urea, 2% CHAPS, citric | Hydrogen bonding, ionic |
| agent | acid to pH 3.0-3.5 | interactions |
| Alkaline dissociating | 9M urea, 2% CHAPS, | Ionic interactions, hydrogen |
| agent | ammonia to pH 11 | bonding |

A preferred elution buffer of the present invention includes a matrix material suitable for use in a mass spectrometer. Inclusion of a matrix material in the buffer, some embodiments of the invention may optionally include eluting analyte(s) from chemical structures directly to mass spectrometer probes, such as protein or biochips. In other embodiments of the invention the matrix may be mixed with analyte(s) after elution from chemical structures. Still other embodiments include eluting analytes directly to SEND or SEAC/SEND protein chips that include an energy absorbing matrix predisposed on the protein chip. In these latter embodiments, there is no need for additional matrix material to be present in the elution buffer.

In one embodiment, separation of the unbound target protein group from the contaminating proteins and target protein group bound to the chemical structures that is coupled to paramagnetic beads is by applying a magnetic force. Proteins bound to the chemical structures/paramagnetic beads will be pulled away from the unbound target protein group. The unbound target protein group will be present in the supernatant from where it can be collected. Paramagnetic beads, typically, comprise a ferromagnetic oxide particle, such as ferromagnetic iron oxide, maghemite, magnetite, or manganese zinc ferrite (see, e.g., U.S. Pat. No. 6,844,426).

### VI. KITS

The present invention also provides kits for purifying a target protein group. The kits contain components that allow one of ordinary skill in the art to perform the methods described herein. In a preferred embodiment, the kit comprises a library of chemical structures having at least 100 different chemical structures and an instruction to purify a target protein group by contacting a sample comprising at least 95% of the target protein group and at most 5% of contaminating proteins with the library of chemical structures.

In another embodiment of the present invention, a kit comprises compositions described herein that are useful for decreasing the range of concentration of analytes in a mixture. In another embodiment, a kit comprises compositions described herein that are useful for detecting analytes in a mixture.

Optionally, a kit of the present invention comprises instructions for the use of the compositions to practice a method of the present invention. The instructions may be present in the subject kits in a variety of forms, one or more of which may be present in the kit. The instruction may be present as printed information on a suitable medium or substrate, e.g., a piece of paper on which, for example, the information of how to purify a target protein group by contacting a sample comprising at least 95% of the target protein group and at most 5% of contaminating proteins with the library of chemical structures, is printed. Another form would be a computer readable medium, such as a CD or diskette on which the information of how to purify a target protein group by contacting a sample comprising at least 95% of the target protein group and at most 5% of contaminating proteins with the library of chemical structures, is recorded. Another form may be a website address that may be used by a user of the kit to access via the interne the information of how to purify a target protein group by contacting a sample comprising at least 95% of the target protein group and at most 5% of contaminating proteins with the library of chemical structures. Other instructions describe the use of compositions in additional methods described herein.

In another embodiment of the present invention, the kits of the present invention further comprise a plurality of containers retaining incubation buffers for contacting the sample with the library of chemical structures or one or more columns, such as fractionating columns.

Kits of the present invention also include a plurality of containers retaining components for sample preparation and analyte isolation. Exemplary components of this nature include one or more wash solutions sufficient for removing unbound material from a particle, and at least one elution solution sufficient to release analyte specifically bound by a chemical structure.

In some kit embodiments of the invention, the library of chemical structures is supplied coupled to a solid support, preferably insoluble beads. In other embodiments, the solid support and library of chemical structures are supplied separately. When supplied separately, the library of chemical structures and/or solid support include a linker moiety and/or a complementary linker moiety that allow the operator of the invention to couple the chemical structures to the solid support during the course of practicing the invention described herein. Kits providing separate library of chemical structures and solid supports may optionally comprise additional reagents necessary to perform the coupling of the library of chemical structures to the solid support.

Furthermore, a kit of this invention can include chromatographic media used to purify the target proteins from a prior sample, for subsequent polishing using the library of chemical structures of this invention.

Additional kit embodiments of the present invention include optional functional components, such as a magnet, that would allow one of ordinary skill in the art to perform any of the method variations described herein.

Although the forgoing invention has been described in some detail by way of illustration and example for clarity and understanding, it will be readily apparent to one ordinary skill in the art in light of the teachings of this invention that certain variations, changes, modifications and substitution of equivalents may be made thereto without necessarily departing from the spirit and scope of this invention. As a result, the embodiments described herein are subject to various modifications, changes and the like, with the scope of this invention being determined solely by reference to the claims appended hereto. Those of skill in the art will readily recognize a variety of non-critical parameters that could be changed, altered or modified to yield essentially similar results.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

As can be appreciated from the disclosure above, the present invention has a wide variety of applications. The invention is further illustrated by the following examples, which are only illustrative and are not intended to limit the definition and scope of the invention in any way.

In a preferred embodiment of this invention, the number of individual chemical structures within a library of chemical structures, for example, a combinatorial library, is so large that it is assumed that each protein present in a sample has an affinity to at least one of the individual chemical structures. Typically, the chemical structures are attached to a solid support, such as beads. When a sample comprising a target protein group of interest that is being purified and a number of contaminating proteins is contacted with such a combinatorial library, individual chemical structure binds to a protein binding partner, including the target protein group and contaminating proteins. The large diversity of the combinatorial library provides chemical structures specific for every protein in a sample, i.e., for the target protein group of interest and the contaminating proteins. However, due to the limited capacity of the beads for a single protein species, minimal amounts of the target protein group will be bound and subsequently be removed from the sample. In theory, if the amount of a diverse combinatorial library attached to beads added to the sample is well calculated, virtually all contaminating proteins should be removed while the target protein group of interest will be very partially removed. The unbound target protein group of interest will remain in the supernatant and can be separated from the proteins bound to the library of chemical structures by filtration, centrifugation or other means. After the separation, the target protein group is collected. The collected target protein group is more pure than the target protein group in the sample.

While it is advantageous to purify a target protein group from a sample comprising the target protein group of interest and contaminating proteins, a skilled artisan will also appreciate that the methods of the invention may also be practiced to purify a target protein group of interest from a sample comprising the target protein group and non-polypeptide contaminants or impurities.

### VII. EXAMPLES

The preparation of magnetic solid phase ligand libraries can be accomplished using two different processes: Using regular beaded sorbent on which a library is constructed and introduce paramagnetic materials afterwards, or making paramagnetic particles first and then construct on the ligand library.
The first approach has been reduced to practice using the following process:
Peptide library beads are packed into a chromatographic column so that to form a bed of about 10 cm long.
The column of beads is equilibrated with a physiological buffer.
One or two volumes of magnetite suspension are pushed through the column bed.
The column is then extensively washed with the initial physiological buffer up to the elimination of the excess of magnetite.
Additional washings are done with solutions currently used for the utilization of the library such as concentrated urea solutions at acidic or alkaline pH, concentrated guanidine-HCl aqueous solutions, thiourea-urea-detergent mixtures, hydro-organic mixtures.

Obtained beads previously carrying peptide ligands have paramagnetic properties and can be separated from liquids by means of a magnetic field. A colloidal suspension of about 100 angstrom magnetite particles (this can be stabilized with an anionic or a cationic surfactant) is slowly loaded from the top of the column.

### Example 1

### Preparation of Magnetic Solid Phase Peptide Ligand Library and Evaluation of Non-Magnetic Solid Phase Peptide Ligand Library and Magnetic Solid Phase Peptide Ligand Library for the Reduction of Protein Concentration Difference in Human Serum (“Equalization”)

In this initial example, the use of hexapeptide libraries on non-magnetic and magnetic particles was evaluated side-by-side to determine if the presence of magnetite has any detrimental effect on using particles with paramagnetic properties in equalization methods. A solid phase ligand library was prepared starting from a pre-existing non-magnetized material like the one described in WO 05094467 A2 (this library was constituted of one peptide type per bead with a terminal primary amino group; “OLOB”). Part of the non-magnetized material was then magnetized as follows. 10 mL of the non-magnetized material having particle diameters between 40 microns and 110 microns was packed in a chromatographic column and washed extensively with a physiological buffer (phosphate buffered saline). The column was then loaded with 20 mL of a magnetic colloidal particle suspension (EMG 807 from Ferrofluidics, Germany) and then left for one hour and washed extensively with the same buffer until excess of magnetic colloidal particles were removed. A second extensive washing was made using a 9M urea comprising citric acid at the final 50 mM concentration. Finally the beads were equilibrated in a physiological buffer. The resulting beads were very susceptible to magnetic field; they could be separated from the liquid supernatant by the simple use of a magnet in few seconds.

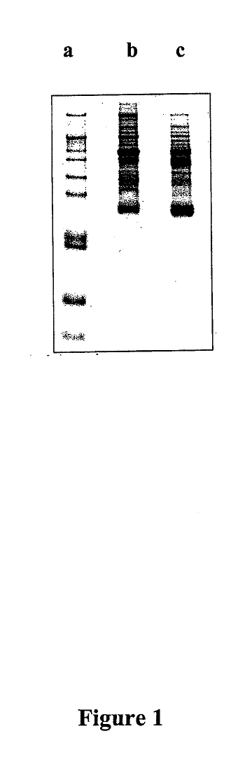

1 mL of these magnetized beads and 1 ml non-magnetized beads, each having attached the hexapeptide library, was then mixed with 10 mL of human serum and left for 30 minutes under gentle agitation. Magnetic peptide combinatorial ligand beads were then separated using a permanent magnet and the supernatant was discarded. The non-magnetized beads were manipulated using standard techniques, such as filtration and centrifugation. After several washing with a physiological buffer, adsorbed proteins on the paramagnetic beads were eluted using 9M urea (at pH 3.3 by citric acid). Collected proteins were then analysed by electrophoresis (SDS-PAGE) and mass spectrometry (SELDI MS) in comparison to the same non-magnetic beads. As can be seen in FIG. 1, both the non-magnetic particles and the particles with paramagnetic properties showed a similar pattern of bound analytes isolated from the hexapeptide libraries attached to either solid support. Further, no significant non-specific binding of analytes to particles with paramagnetic properties was observed.

### Example 2

### Preparation and Evaluation of Magnetic Solid Phase Peptide Ligand Library for the Reduction of Protein Concentration Difference in Human Serum

1 mL of reactive particles with paramagnetic properties of 1 μm diameter (from Dynal) suspended in 2 ml volume of solution, were separated from the supernatant using a magnetic bar and then washed several times with 100 mM sodium borate, pH 9.5. Separately 60 mg of combinatorial hexapeptides were dissolved in a mixture composed of 3 mL of 100 mM sodium borate, pH 9.5, 1.3 mL of ethanol and 1 mL of DMSO. The conditioned settled particles with paramagnetic properties (1 mL) were added to the hexapeptide peptide solution. Then 2.75 mL of 3.0 M ammonium sulphate in 100 mM sodium borate, pH 9.5 were added. The mixture was incubated at 37° C. for 25 hour under gentle shaking.

While the beads were maintained inside the vessel due to applying a magnetic field, the supernatant was replaced with a physiological buffer containing 0.1 M ethanolamine to cap any remaining active groups. This end-capping operation was done overnight at 37° C. Finally the resulting coupled beads were rinsed extensively with a physiological buffer until total elimination of reagents and by-products. The library generated comprised all peptides on a single bead (“ALOB”, all-ligands-one-bead) having a free terminal carboxyl group.

The resultant combinatorial peptide library on the particles with paramagnetic properties was evaluated as described in the Example 1. Briefly, 80 μL of these magnetized beads were mixed with 800 μL of human serum and left for 30 minutes under gentle agitation. Magnetic peptide combinatorial ligand beads were then separated using a permanent magnet and the supernatant discarded. After several washing with a physiological buffer adsorbed proteins on the beads were eluted using a 9M urea at pH 3.3 by citric acid. Collected serum proteins were then analyzed by electrophoresis (SDS-PAGE) and mass spectrometry (SELDI MS).

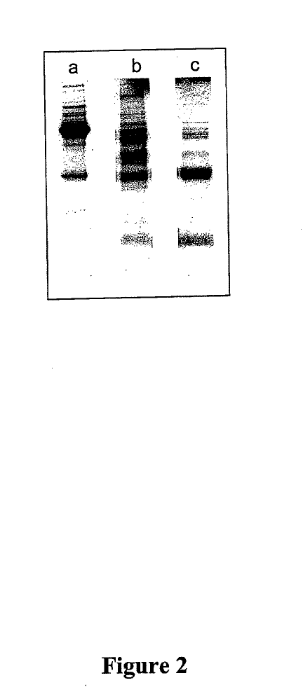

Experimental results shown in FIG. 2 demonstrated that similar serum proteins are captured on the 1 μm diameter magnetic beads (lane c) than those captured on the larger size beads (FIG. 1, lane c) or with non-magnetic beads (FIG. 1, lane b, FIG. 2, lane b). Again, as observed for larger magnetic beads, no significant non-specific binding was observed on the 1 μm diameter magnetic beads.

### Example 3

### Reproducibility of Sample Treatment with Particles with Paramagnetic Properties Carrying a Peptide Ligand Library

Magnetic 1 μm diameter beads coated with combinatorial peptide ligands from Example 2 were the used for a comparative study to check the reproducibility of serum treatment.

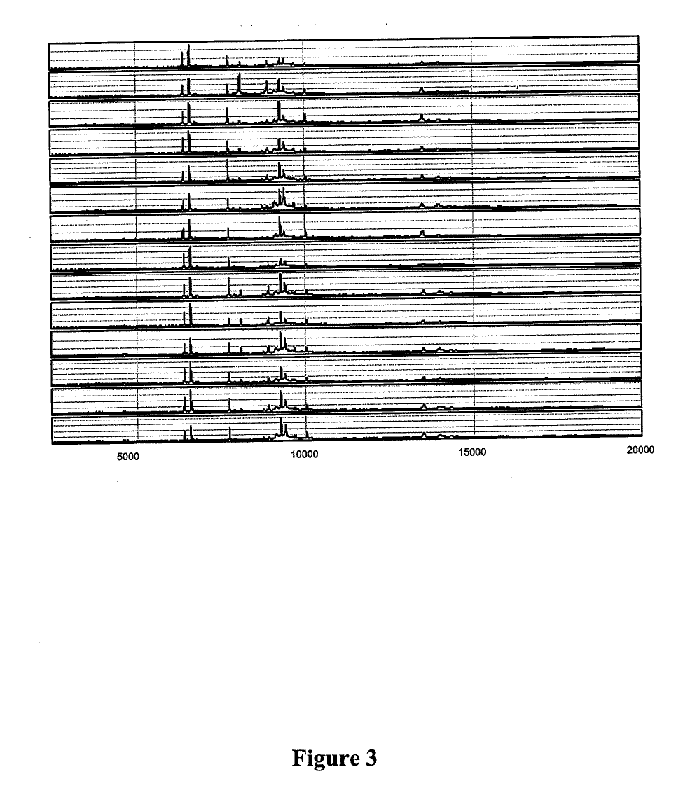

14 times 10 μL of beads were taken from the stock suspension and dispensed in 14 different small tubes. To each tube 800 μL of serum was added and all tubes incubated for 30 minutes under gentle agitation. Supernatants of each tube were separated as described above in Examples 1 and 2 and washed extensively with a physiological buffer. Adsorbed proteins on beads from each tube were then eluted using an aqueous solution of 9M urea containing 50 mM citric acid, pH 3.3. Collected protein solutions were then analyzed by SELDI MS. FIG. 3 shows the good reproducibility of this analysis.

### Example 4

### Preparation and Evaluation of Magnetic Solid Phase Peptide Ligand Library for the Reduction of Protein Concentration Difference in Human Serum (“Equalization”)

Reactive particles with paramagnetic properties of 2.8 μm diameter from Dynal are modified so that to introduce primary amines. This is accomplished according to the recommendation of the supplier for the coupling of ethylene diamine. The aminated derivative is washed extensively with phosphate buffered saline and then with deionised water. The obtained derivative is then washed progressively with dimethyllformamide several times to completely eliminate water. At this stage the beads are used for the solid phase peptide synthesis under classical combinatorial manner (split-couple-and-recombine) to get a final hexapeptide library. This library has a terminal primary amine. All manipulations such as solid-liquid separations are done using external magnetic field to maintain beads inside the vessel.

The final product is extensively washed with a sequence of solutions: 100% DMF, 50%-50% DMF-water, 100% water, physiological buffer and finally stored in 1M sodium chloride solution containing 20% ethanol. The final suspension is then stored at +4° C. The library constituted in this way comprises one peptide type per bead with a terminal primary amino group.

20 μL of bead suspension containing about 10 μL settled particles with paramagnetic properties are washed extensively washed with a physiological buffer and added to 200 μL of human serum. The suspension is shaken for 30 minutes at room temperature. From the suspension, particles with paramagnetic properties are removed by means a small magnet and introduced into a small tube and washed until unbound proteins were removed from the supernatant. Beads with captured proteins from serum are then treated with an elution buffer composed of 9M urea acidified at pH 3.3 by addition of 2M sodium citrate. Under these conditions captured proteins are desorbed from the beads and collected separately. Recovered proteins are then analyzed by SDS-PAGE and SELDI MS as described herein. Results are expected to show that protein composition is similar to the initial sample; however, many more protein species are expected to be detected as a result of the reduction of concentration difference of proteins in the initial sample.

### INCORPORATION BY REFERENCE

All publications, patents and patent applications cited in this specification are herein incorporated in their entirety by reference as if each individual publication, patent or patent application were specifically and individually indicated to be incorporated by reference.

What is claimed is:

1. A method of making a combinatorial library of diverse chemical structures bound to particles comprising performing a number of rounds of split-couple-and-recombine chemical synthesis with a collection of particles with paramagnetic properties having a diameter between about 100 nm and about 10 microns and a plurality of different chemical moieties, wherein each round of the split-couple-and-recombine chemical synthesis adds a chemical moiety to the chemical structure, and involves magnetically manipulating the particle with paramagnetic properties, and wherein the number of rounds suffices to assemble a library having a diversity of at least 100,000 unique chemical structures.

2. The method of claim 1 wherein the particles with paramagnetic properties have a diameter between about 300 nm and about 5 microns.

3. The method of claim 1 wherein the particles with paramagnetic properties have a diameter between about 1 micron and 3 microns.

4. The method of claim 1 wherein the chemical structures are peptides, oligonucleotides, oligosaccharides or synthetic organic molecules and the library has a diversity of at least 1 million unique chemical structures.

5. The method of claim 1 wherein the chemical structures are peptides and the library has a diversity of at least 3 million unique peptides.

6. The method of claim 1 wherein the chemical structures are peptides and the library has a diversity of at least 64 million unique peptides.

7. The method of claim 1 wherein the library has a size of at least 100,000,000 chemical structures.

8. The method of claim 1 wherein the library comprises substantially all of the members of a combinatorial library.

9. The method of claim 1 wherein the volume of the library is less than about 100 microliters.

10. The method of claim 1 wherein the particles with paramagnetic properties comprise a polymeric material with a paramagnetic material embedded therein.

11. The method of claim 1 wherein the particles with paramagnetic properties comprise porous particles wherein a paramagnetic material is lodged in the porous particles.

12. A method for decreasing the range of concentrations of different analyte species in a mixture comprising the steps of:
(a) providing a first sample comprising a plurality of different analyte species present in the first sample in a first range of concentrations;
(b) contacting the first sample with an amount of a library of diverse chemical structures bound to a collection of particle with paramagnetic properties having a diameter between about 100 nm and about 10 microns, wherein the chemical structures comprise a plurality of different chemical moieties and the chemical structures bound to each individual particle with paramagnetic properties have substantially the same structure and the combinatorial library has a diversity of at least 100,000 unique chemical structures;
(c) capturing amounts of the different analyte species from the first sample with the different chemical structures and removing unbound analyte species; and
(d) isolating the captured analyte species from the chemical structures to produce a second sample comprising a plurality of different analyte species present in the second sample in a second range of concentrations;
wherein the amount of the library is selected to capture amounts of the different analyte species so that the second range of concentrations is less than the first range of concentrations.

13. The method of claim 12 wherein isolation comprises a step-wise elution to produce a plurality of aliquots.

14. The method of claim 12 further comprising the step of detecting the isolated analytes.

15. The method of claim 14 wherein the isolated analytes are detected by mass spectrometry or electrophoresis.

16. The method of claim 12 wherein isolating comprises eluting the analytes from the particles onto a biochip with an adsorbent surface, wherein the adsorbent surface binds analytes from the eluate.

17. A method for detecting analytes in a mixture comprising the steps of:
(a) providing a first sample comprising a plurality of different analyte species present in the first sample in a first range of concentrations;
(b) contacting the first sample with an amount of a library of diverse chemical structures bound to a collection of particles with paramagnetic properties having a diameter between about 100 nm and about 10 microns, wherein the chemical structures comprise a plurality of different chemical moieties and the chemical structures bound to each individual particle with paramagnetic properties have substantially the same structure and the combinatorial library has a diversity of at least 100,000 unique chemical structures;
(c) capturing amounts of the different analyte species from the first sample with the different chemical structures and removing unbound analyte species;
(d) placing the particles with captured analytes into a mass spectrometer; and
(e) detecting the captured analytes by laser desorption mass spectrometry.

18. A method for purifying a target protein group comprising the steps of:
(a) contacting a sample comprising at least 95% of the target protein group and at most 5% of contaminating proteins with a library of diverse chemical structures bound to a collection of particle with paramagnetic properties having a diameter between about 100 nm and about 10 microns, wherein the chemical structures comprise a plurality of different chemical moieties and the chemical structures bound to each individual particle with paramagnetic properties have substantially the same structure and the combinatorial library has a diversity of at least 100,000 unique chemical structures in an amount sufficient to bind contaminating proteins and a minority of the target protein group;
(b) binding the contaminating proteins and the minority of the target protein group to the library of chemical structures;
(c) separating the unbound target protein group from the contaminating proteins and target protein group bound to the library of chemical structures; and
(d) collecting the unbound target protein group from the sample, whereby the collected target protein group is more pure than the target protein group in the sample.
